(19) United States
(12) Patent Application Publication
(10) Pub. No.: US 2026/0102213 A1
(43) Pub. Date: Apr. 16, 2026

(54) RELATIVE KINEMATIC COMPENSATION FOR ROBOTIC ARMS BASED ON FEEDBACK FROM FORCES TRANSMITTED BY MEDICAL INSTRUMENTS

(71) Applicant: AURIS HEALTH, INC., Santa Clara, CA (US)

(72) Inventors: Michael David Leslie, San Francisco, CA (US); Yusra Farhat Ullah, Falcon Heights, MN (US); Tara Olyaee Hammond, Mountain View, CA (US); Jackson Arthur Dibachi, Los Altos, CA (US); Cole Clampffer, San Mateo, CA (US)

(73) Assignee: AURIS HEALTH, INC., Santa Clara, CA (US)

(21) Appl. No.: 18/916,613

(22) Filed: Oct. 15, 2024

(51) Int. Cl. A61B 34/30; A61B 90/00; B25J 9/16

(52) U.S. Cl. CPC A61B 34/30; B25J 9/1633; A61B 2090/066; A61B 2090/0811

(57) ABSTRACT

This disclosure provides methods, devices, and systems for calibrating robotic systems. The present implementations more specifically relate to techniques for correcting relative misalignment between robotic arms by iteratively adjusting a kinematic model based on feedback from forces or torque exerted on the robotic arms in response to manipulating medical instruments coupled thereto. More specifically, a controller may move the robotic arms through a series of poses that cause a first instrument coupled to a first robotic arm to be driven inside a lumen of a second instrument coupled to a second robotic arm. The controller may determine a baseline torque on one or more of the robotic arms by moving the arms through the same series of poses, but without instruments coupled thereto, and may selectively adjust the kinematic model based on differences between the feedback torque on the robotic arms and the baseline torque on the corresponding arms.

### TECHNICAL FIELD

This disclosure relates generally to medical systems, and specifically to relative kinematic compensation for robotic arms based on feedback from forces transmitted by medical instruments.

### DESCRIPTION OF RELATED ART

Many medical procedures, such as bronchoscopy, laparoscopy, ureteroscopy, or percutaneous nephrolithotomy (PCNL), involve a series of complex steps that require careful movement and positioning of medical tools or instruments inside a patient's body. Some medical procedures can now be performed, at least in part, by a robotic system or apparatus, which can aid a user or operator (such as a physician or technician) in navigating or positioning such instruments. For example, the user may control or operate one or more robotic arms to manipulate medical instruments during a medical procedure. Kinematic models are often used in robotics to describe relationships between the positions, velocities, and accelerations of various joints and linkages (such as robotic arm sections) of each robotic arm and/or the instruments (or end effectors) coupled thereto. However, such kinematic models are often idealized models that may not account for real-world variations affecting the deployment of robotic arms, such as process variations (in manufacturing), mounting inaccuracies, and temperature changes, among other examples. As a result, a user commanded pose based on a kinematic model may differ from the actual robotic arm pose achieved in response to such commands, which can lead to relative misalignment between multiple robotic arms.

Some medical instruments (such as an access sheath) are designed to be used in close proximity and coordination with other medical instruments (such as a scope or catheter) such that relative misalignments between the robotic arms can cause the medical instruments to make unwanted contact or otherwise exert undesired forces against one another. Such forces can result in damage to the instruments or overexertion of various motors or actuators in the robotic arms attempting to maintain a target joint configuration. Thus, there is a need to compensate or correct for relative misalignment between robotic arms associated with a medical system.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method performed by a robotic system. The method includes steps of moving a first robotic arm through a plurality of first poses based on a kinematic model while a first instrument is coupled to the first robotic arm, where the plurality of first poses cause the first instrument to be driven inside a lumen of a second instrument coupled to a second robotic arm; measuring a torque on one or more motors of the first robotic arm in response to moving the first robotic arm through the plurality of first poses; moving the first robotic arm through a plurality of second poses based on the kinematic model while the first instrument is decoupled from the first robotic arm; measuring a baseline torque on the one or more motors of the first robotic arm in response to moving the first robotic arm through the plurality of second poses; determining whether the first robotic arm and the second robotic arm are misaligned based at least in part on the measured torque, and the baseline torque, on the one or more motors of the first robotic arm; and selectively adjusting the kinematic model based on whether the first robotic arm and the second robotic arm are misaligned.

Another innovative aspect of the subject matter of this disclosure can be implemented in a controller for a robotic system, including a processing system and a memory. The memory stores instructions that, when executed by the processing system, cause the controller to move a first robotic arm through a plurality of first poses based on a kinematic model while a first instrument is coupled to the first robotic arm, where the plurality of first poses cause the first instrument to be driven inside a lumen of a second instrument coupled to a second robotic arm; measure a torque on one or more motors of the first robotic arm in response to moving the first robotic arm through the plurality of first poses; move the first robotic arm through a plurality of second poses based on the kinematic model while the first instrument is decoupled from the first robotic arm; measure a baseline torque on the one or more motors of the first robotic arm in response to moving the first robotic arm through the plurality of second poses; determine whether the first robotic arm and the second robotic arm are misaligned based at least in part on the measured torque, and the baseline torque, on the one or more motors of the first robotic arm; and selectively adjust the kinematic model based on whether the first robotic arm and the second robotic arm are misaligned.

### BRIEF DESCRIPTION OF THE DRAWINGS

The present implementations are illustrated by way of example and are not intended to be limited by the figures of the accompanying drawings.

FIG. 1 shows an example medical system for performing a robotic-assisted medical procedure, according to some implementations.

FIG. 2 shows a more detailed example of the robotic system depicted in FIG. 1, according to some implementations.

FIG. 3 shows an example arrangement of the medical system of FIG. 1 for performing another medical procedure, according to some implementations.

FIG. 4 shows an example arrangement of the medical system of FIG. 1 for performing another medical procedure, according to some implementations.

FIG. 5 shows an example instrument driver, according to some implementations.

FIG. 6 shows an example medical instrument with a paired instrument driver, according to some implementations.

FIG. 7 shows another example medical instrument with a paired instrument driver, according to some implementations.

FIG. 8 shows a block diagram of an example localization system, according to some implementations.

FIG. 9 shows another example robotic system, according to some implementations.

FIG. 10A shows an example configuration of the robotic system of FIG. 9 in which the robotic arms are misaligned.

FIG. 10B shows another example configuration of the robotic system of FIG. 9 in which the robotic arms are misaligned.

FIG. 11 shows a timing diagram depicting an example insertion and retraction of medical instruments coupled to respective robotic arms.

FIG. 12 shows an illustrative flowchart depicting an example process for iteratively adjusting a kinematic model to compensate for relative misalignment of robotic arms.

FIG. 13A-13H show example configurations of the robotic system of FIG. 9 for determining and correcting relative misalignment between the robotic arms.

FIG. 14 shows another timing diagram depicting an example insertion and retraction of medical instruments coupled to robotic arms.

FIG. 15 shows a block diagram depicting an example controller for a robotic system, according to some implementations.

FIG. 16 shows an illustrative flowchart depicting an example operation for correcting relative misalignment between robotic arms, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations.

Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, some medical procedures can now be performed, at least in part, by a robotic system or apparatus, which can aid a user or operator (such as a physician or technician) in navigating or positioning medical instruments. Some medical instruments (such as an access sheath) are designed to be used in close proximity and coordination with other medical instruments (such as a scope or catheter) such that relative misalignments between robotic arms can cause the medical instruments to make unwanted contact or otherwise exert undesired forces against one another. For example, during a bronchoscopy, an endoscope can be inserted and retracted within a lumen of an access sheath so that the instruments are coaxial with one another while being driven by respective robotic arms. To preserve the coaxiality of such instruments, the end effectors and/or instrument device manipulators (IDMs) of the robotic arms may be configured to move along a “virtual rail.” However, because each instrument is coupled to and manipulated by a respective robotic arm, relative misalignment between the arms can result in loss of coaxiality between the instruments thereby causing the outer surface of the endoscope to press or rub against the inner surface of the access sheath as the instruments are driven in relation to one another. Such forces can cause damage to the instruments and/or overexertion of various motors or actuators in the robotic arms.

The relative misalignment between robotic arms also causes the instruments to exert torque or other counterforces against the motors or actuators in the robotic arms. Aspects of the present disclosure recognize that such forces can be detected and/or measured by the motors themselves, or by various sensors in a robotic arm, and further used to guide realignment of the robotic arms. For example, each IDM may include one or more torque sensors that are configured to measure torque on the IDM. The measured torque (τ) is equal to the sum of any torque (τ_{D}) exerted by the drive motor(s) internal to the IDM (also referred to as “drive torque”) and any counter-torque (τ_{M}) exerted by the instrument coupled to the IDM (also referred to as “misalignment torque”). More specifically, the misalignment torque τ_{M} can be determined as a function of the measured torque τ and the drive torque τ_{D}, where τ_{M}=τ−τ_{D}. In some aspects, the robotic system may determine whether the misalignment torque τ_{M} is outside a normal or expected range (associated with driving instruments that are coaxially aligned) so as to indicate relative misalignment between the robotic arms.

Existing techniques for correcting robotic arm misalignment rely on additional sensing modalities (such as cameras) to track the poses of the arms in three-dimensional (3D) space and update a kinematic model. However, such sensor technologies are often expensive to implement and add little or no utility to the functioning of the robotic system beyond calibration of the robotic arms. Aspects of the present disclosure recognize that the degree and magnitude of the misalignment torque τ_{M} can guide the selection of a kinematic offset to be applied to one or more robotic arms to correct the misalignment between the arms. For example, the robotic system may iteratively adjust a kinematic model so that at least one of the robotic arms has a new position and/or orientation when operated in relation to the other robotic arm (such as along a virtual rail). For each iteration, the robotic system may determine whether the adjustments to the kinematic model result in lowering the misalignment torque τ_{M}. As such, the robotic system may iteratively adjust the kinematic model until the misalignment torque τ_{M} is minimized and/or falls within a threshold range.

Various aspects relate generally to systems and techniques for calibrating robotic systems, and more particularly, to correcting relative misalignment between robotic arms by iteratively adjusting a kinematic model based on feedback from forces or torque exerted on the robotic arms in response to manipulating instruments in accordance with a medical procedure. For example, a controller may move a first robotic arm and a second robotic arm through a series of poses that cause a first instrument (such as a scope or catheter) coupled to the first robotic arm to be driven (such as by insertion or retraction) inside a lumen of a second instrument (such as an access sheath) coupled to the second robotic arm. In some aspects, the controller may measure a baseline torque on one or more of the robotic arms by moving the arms through the same series of poses, but without any instruments coupled thereto, and may selectively adjust the kinematic model based on differences between the feedback torque on the robotic arms and the baseline torque on the corresponding arms. More specifically, the controller may iteratively adjust the kinematic model until the difference between the feedback torque and the baseline torque on the robotic arms is less than or equal to a threshold amount.

Particular implementations of the subject matter described in this disclosure can be implemented to realize one or more of the following potential advantages. By comparing the baseline torque with the feedback torque, aspects of the present disclosure can separate the amount of torque needed to overcome any inherent friction (or “stickiness”) in the robotic arm joints from the additional torque needed to drive an instrument. Because the baseline torque represents the drive torque τ_{D} associated with the series of robotic arm poses, the controller may determine the misalignment torque τ_{M} as the difference between the feedback torque (τ) and the baseline torque τ_{D}. By iteratively adjusting the kinematic model to reduce and/or minimize the misalignment torque τ_{M}, aspects of the present disclosure can correct relative misalignments between the robotic arms using only the feedback from torque sensors and/or motors in the arms. Many existing robotic systems rely on such feedback to maintain target joint configurations. Thus, aspects of the present disclosure can leverage the existing hardware of a robotic system to correct for relative misalignment between robotic arms in a much more cost-effective manner (compared to existing techniques that rely on additional sensing modalities, such as cameras).

Aspects of the present disclosure may be used to perform robotic-assisted medical procedures, such as endoscopic access, percutaneous access, or treatment for a target anatomical site. For example, robotic tools may engage or control one or more medical instruments (such as an endoscope and/or a percutaneous access catheter) to access a target site within a patient's anatomy or perform a treatment at the target site. In some implementations, the robotic tools may be guided or controlled by a physician. In some other implementations, the robotic tools may operate in an autonomous or semi-autonomous manner. The systems and techniques described herein also may be applicable beyond the context of medical procedures (such as in simulated environments or laboratory settings, such as with models or simulators, among other examples).

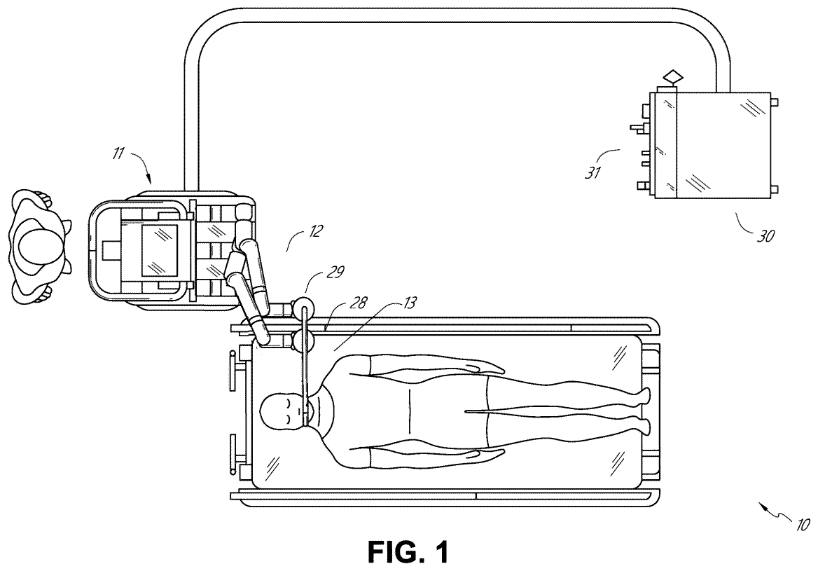

FIG. 1 shows an example medical system 10 for performing a robotic-assisted medical procedure, according to some implementations. For example, during a bronchoscopy, the system 10 may includer a cart 11 having one or more robotic arms 12 (to deliver a medical instrument, such as a steerable endoscope 13, which may be a procedure-specific bronchoscope for bronchoscopy, to a natural orifice access point (such as the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures.

With continued reference to FIG. 1, once the cart 11 is properly positioned, the robotic arms 12 may insert the steerable endoscope 13 into an anatomy, for example, of a patient, robotically, manually, or a combination thereof. As shown, the steerable endoscope 13 may include at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the anatomy. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius.

The use of separate instrument drivers 28 also allows the leader portion and sheath portion to be driven independent of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments may need to be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician or technician. Additionally, the division of functionality between the cart or table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive or solid state drive. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to system that may be deployed through the endoscope 13. These components may also be controlled using the computer system of tower 30. In some implementations, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include opto-electronics equipment for detecting, receiving, and processing data received from the optical sensors or cameras throughout the robotic system 10. In combination with the control system, such opto-electronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system such as, for example, a console mounted on top of the cart.

The console 31 may include a user interface and a display screen, such as a touchscreen, for the physician operator. Consoles in system 10 are generally designed to provide both robotic controls as well as pre-operative and real-time information of the procedure, such as navigational and localization information of the endoscope 13. When the console 31 is not the only console available to the physician, it may be used by a second operator, such as a nurse, to monitor the health or vitals of the patient and the operation of system, as well as provide procedure-specific data, such as navigational and localization information.

The tower 30 may be coupled to the cart 11 and endoscope 13 through one or more cables or connections (not shown). In some implementations, the support functionality from the tower 30 may be provided through a single cable to the cart 11, simplifying and de-cluttering the operating room. In some other implementations, specific functionality may be coupled in separate cabling and connections. For example, while power may be provided through a single power cable to the cart, the support for controls, optics, fluidics, and/or navigation may be provided through a separate cable.

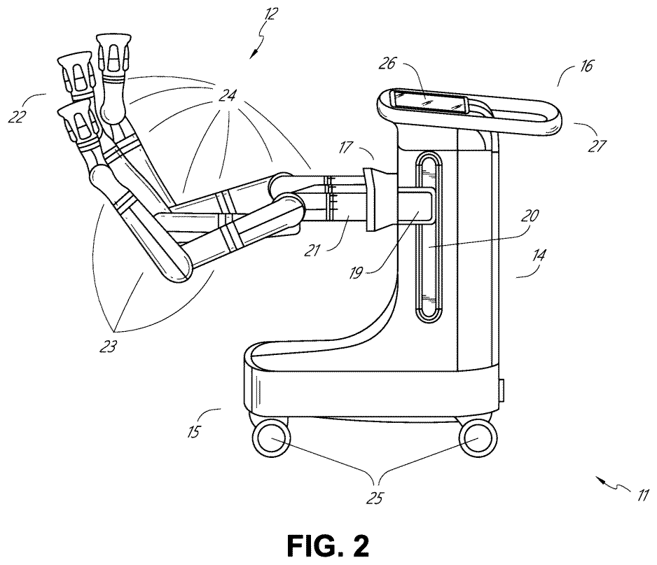

FIG. 2 shows a more detailed example of the robotic system depicted in FIG. 1, according to some implementations. With reference to FIG. 2, cart 11 may include an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of robotic arms 12 to be angled in a variety of configurations.

In some implementations, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally include mechanisms, such as gears and motors, that are designed to use a vertically aligned lead screw to translate the carriage 17 in a mechanized fashion in response to control signals generated in response to user inputs, such as inputs from the console 16.

The robotic arms 12 may generally include robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm. In the example shown, each of the arms 12 have seven joints, and thus provide seven degrees of freedom. In actual implementations, one or more of the arms 12 may have a different number of joints and/or linkages, with the same or different configurations, compared to what is shown in FIG. 2 and/or compared to other arms 12 of the robotic system. A multitude of joints result in a multitude of degrees of freedom, allowing for “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and arms 12 over the floor. Accordingly, the cart base 15 houses heavier components, such as electronics, motors, power supply, as well as components that either enable movement and/or immobilize the cart. For example, the cart base 15 includes rollable wheel-shaped casters 25 that allow for the cart to easily move around the room prior to a procedure. After reaching the appropriate position, the casters 25 may be immobilized using wheel locks to hold the cart 11 in place during the procedure.

Positioned at the vertical end of column 14, the console 16 allows for both a user interface for receiving user input and a display screen (or a dual-purpose device such as, for example, a touchscreen 26) to provide the physician user with both pre-operative and intra-operative data. Potential pre-operative data on the touchscreen 26 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 16 may be positioned and tilted to allow a physician to access the console from the side of the column 14 opposite carriage 17. From this position the physician may view the console 16, robotic arms 12, and patient while operating the console 16 from behind the cart 11. As shown, the console 16 also includes a handle 27 to assist with maneuvering and stabilizing cart 11.

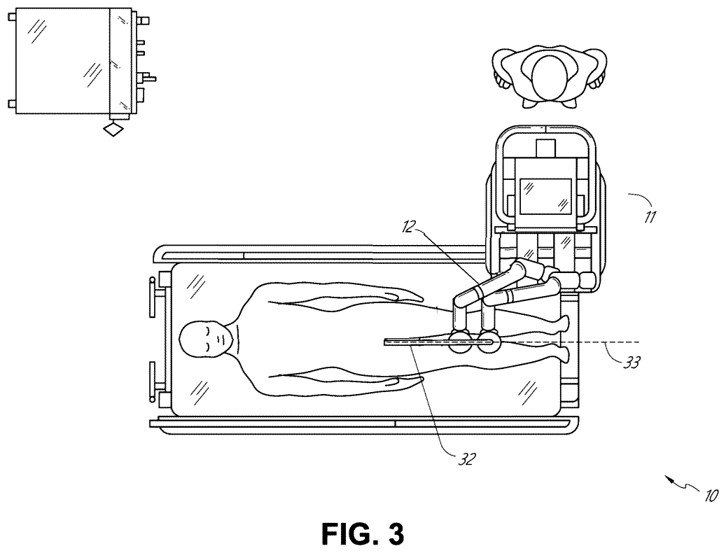

FIG. 3 shows an example arrangement of the medical system 10 of FIG. 1 for performing another medical procedure, according to some implementations. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using laser or ultrasonic lithotripsy device deployed down the working channel of the ureteroscope 32. After lithotripsy is complete, the resulting stone fragments may be removed using baskets deployed down the ureteroscope 32.

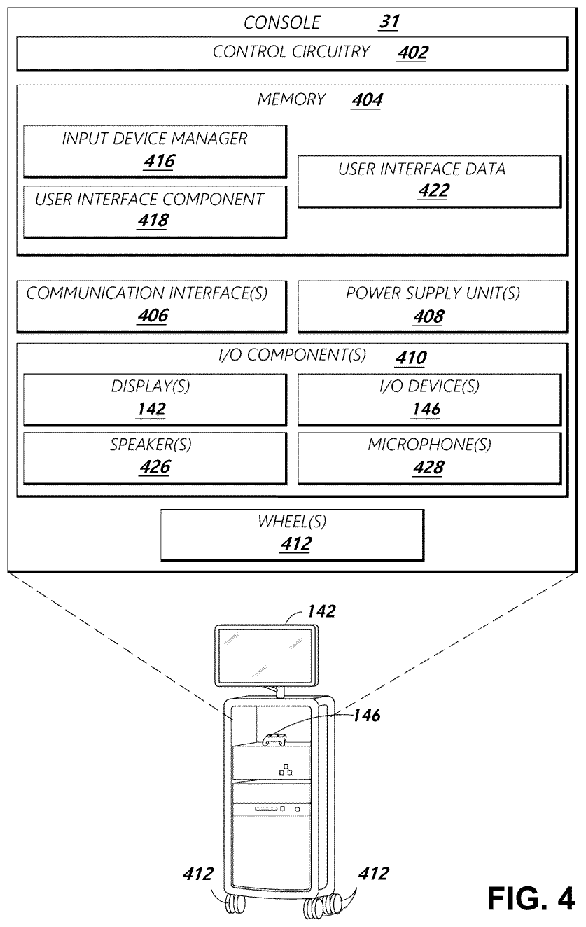

FIG. 4 shows a more detailed example of the console 31 of FIG. 1, according to some implementations. As shown in FIG. 4, the console 31 can include one or more devices, modules, and/or units (also referred to as “components”), either separately or individually and/or in combination or collectively: control circuitry 402, data storage or memory 404, one or more communication interfaces 406, one or more power supply units 408, one or more I/O components 410, and/or one or more wheels 412 (such as casters or other types of wheels). In the example of FIG. 4, the console 31 is illustrated as a cart-based system that is movable with one or more wheels 412. After reaching the appropriate position, the one or more wheels 412 can be immobilized using wheel locks to hold the console 31 in place. However, the console 31 also can be implemented as a stationary system, integrated into another system and/or device.

Although certain components of the console 31 are illustrated in FIG. 4, it should be understood that additional components not shown can be included in implementations in accordance with the present disclosure. Furthermore, certain of the illustrated components can be omitted in some implementations. Although the control circuitry 402 is illustrated as a separate component in the diagram of FIG. 4, it should be understood that any or all of the remaining components of the console 31 can be embodied at least in part in the control circuitry 402. That is, the control circuitry 402 can include various devices (active and/or passive), semiconductor materials and/or areas, layers, regions, and/or portions thereof, conductors, leads, vias, connections, and/or the like, wherein one or more of the other components of the console 31 and/or portion(s) thereof can be formed and/or embodied at least in part by such circuitry components and/or devices.

The various components of the console 31 can be electrically and/or communicatively coupled using certain connectivity circuitry, devices, and/or features, which may or may not be part of the control circuitry 402. For example, the connectivity feature(s) can include one or more printed circuit boards configured to facilitate mounting and/or interconnectivity of at least some of the various components or circuitry of the console 31. In some implementations, two or more of the control circuitry 402, the data storage or memory 404, the communication interface(s) 406, the power supply unit(s) 408, and/or the I/O component(s) 410, can be electrically and/or communicatively coupled to each other.

As illustrated, the memory 404 can include an input device manager 416 and a user interface component 418 configured to facilitate various functionality discussed herein. In some implementations, the input device manager 416, and/or the user interface component 418 can include one or more instructions that are executable by the control circuitry 402 to perform one or more operations. Although many implementations are discussed in the context of the components 416-418 including one or more instructions that are executable by the control circuitry 402, any of the components 416-418 can be implemented at least in part as one or more hardware logic components, such as one or more application specific integrated circuits (ASIC), one or more field-programmable gate arrays (FPGAs), one or more program-specific standard products (ASSPs), one or more complex programmable logic devices (CPLDs), and/or the like. Furthermore, although the components 416-418 are illustrated as being included within the console 31, any of the components 416-418 can be implemented at least in part within another device/system, such as the cart 11 of FIGS. 1-3. Similarly, any of the other components of the console 31 can be implemented at least in part within another device/system.

The input device manager 416 can be configured to receive inputs from an input device 146 and translate the inputs into actions performable by the robotic system. The user interface component 418 can be configured to facilitate one or more user interfaces (also referred to as a “graphical user interface” or “GUI”). For example, the user interface component 418 can provide user interface data 422 for display to the user. The communication interfaces 406 can be configured to communicate with one or more devices, sensors, and/or systems. For example, the one or more communication interfaces 406 can send and/or receive data in a wireless and/or wired manner over a network. A network in accordance with implementations of the present disclosure can include a LAN, WAN (such as the Internet), PAN, or body area network (BAN), among other examples. In some implementations, the one or more communication interfaces 406 can implement a wireless technology such as Bluetooth, Wi-Fi, or near field communication (NFC), among other examples.

The one or more power supply units 408 can be configured to manage power for the console 31 and/or the cart 11. In some implementations, the one or more power supply units 408 include one or more batteries, such as a lithium-based battery, a lead-acid battery, an alkaline battery, and/or another type of battery. That is, the one or more power supply units 408 can comprise one or more devices and/or circuitry configured to provide a source of power and/or provide power management functionality. Moreover, in some implementations the one or more power supply units 408 include a mains power connector that is configured to couple to an alternating current (AC) or direct current (DC) mains power source.

The one or more I/O components 410 can include a variety of components to receive input and/or provide output, such as to interface with a user. The one or more I/O components 410 can be configured to receive touch, speech, gesture, or any other type of input. In examples, the one or more I/O components 410 can be used to provide input regarding control of a device or system, such as to control the robotic system and/or navigate a medical instrument coupled to the robotic system. As shown, the one or more I/O components 410 can include the one or more displays 142 (also referred to as “display devices”) configured to display data. The one or more displays 142 can include one or more liquid-crystal displays (LCD), light-emitting diode (LED) displays, organic LED displays, plasma displays, electronic paper displays, and/or any other type(s) of technology. In some implementations, the one or more displays 142 include one or more touchscreens configured to receive input and/or display data. Further, the one or more I/O components 410 can include the one or more input devices 146, which can include a touchscreen, touch pad, controller, mouse, keyboard, wearable device (such as an optical head mounted display), or virtual or augmented reality device (such as head mounted display). Additionally, the one or more I/O components 410 can include one or more speakers 426 configured to output sounds based on audio signals and/or one or more microphones 428 configured to capture or record audio. In some implementations, the one or more I/O components 410 include or are implemented as a console.

Although not shown in FIG. 4, the console 31 can include and/or control other components, such as one or more pumps, flow meters, valve controls, and/or fluid access components in order to provide controlled irrigation and/or aspiration capabilities to a medical instrument (such as a scope) and/or a device that can be deployed through a medical instrument. In some implementations, irrigation and aspiration capabilities can be delivered directly to a medical instrument through separate cable(s). Further, the console 31 can include a voltage and/or surge protector designed to provide filtered and/or protected electrical power to another device, such as the cart 11, thereby avoiding placement of a power transformer and other auxiliary power components in cart 11, resulting in a smaller, more moveable cart 11.

The console 31 can also include support equipment for sensors deployed throughout the robotic system. For example, the console 31 can include opto-electronics equipment for detecting, receiving, and/or processing data received from optical sensors and/or cameras. Such opto-electronics equipment can be used to generate real-time images for display in any number of devices/systems, including in the console 31.

In some implementations, the console 31 can be coupled to the cart 11 and/or a medical instrument, such as the medical instrument 13, through one or more cables or connections (not shown). In some implementations, support functionality from the console 31 can be provided through a single cable, simplifying and de-cluttering an operating room. In other implementations, specific functionality can be coupled in separate cabling and connections. For example, while power can be provided through a single power cable, the support for controls, optics, fluidics, and/or navigation can be provided through a separate cable.

The term “control circuitry” is used herein according to its broad and ordinary meaning, and can refer to any collection of one or more processors, processing circuitry, processing modules or units, chips, dies (such as semiconductor dies including come or more active and/or passive devices and/or connectivity circuitry), microprocessors, micro-controllers, digital signal processors, microcomputers, central processing units, graphics processing units, field programmable gate arrays, programmable logic devices, state machines (such as hardware state machines), logic circuitry, analog circuitry, digital circuitry, and/or any device that manipulates (analog and/or digital) signals based on hard coding of the circuitry and/or operational instructions. Control circuitry can further comprise one or more, storage devices, which can be embodied in a single memory device, a plurality of memory devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. It should be noted that in implementations in which control circuitry comprises a hardware state machine (and/or implements a software state machine), analog circuitry, digital circuitry, and/or logic circuitry, data storage device(s) or register(s) storing any associated operational instructions can be embedded within, or external to, the circuitry comprising the state machine, analog circuitry, digital circuitry, and/or logic circuitry.

The term “memory” is used herein according to its broad and ordinary meaning and can refer to any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s) or protocols, including any suitable or desirable computer-readable instructions, data structures, program modules, or other types of data.

Computer-readable media that can be implemented in accordance with implementations of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

The term “robotic manipulator” is used herein according to its broad and ordinary meaning and may refer to any type or configuration of one or more robotic end effectors, actuators, gears, drives, rails, interfaces, or the like. The end effectors of the system's robotic arms include (i) an instrument driver (alternatively referred to as “instrument drive mechanism” or “instrument device manipulator” (IDM)) that incorporate electro-mechanical means for actuating the medical instrument and (ii) a removable or detachable medical instrument which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

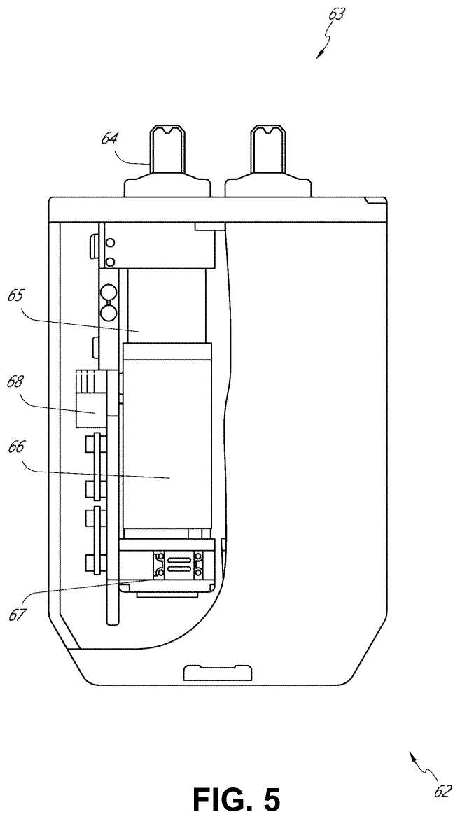

FIG. 5 shows an example instrument driver 62, according to some implementations. Positioned at the distal end of a robotic arm, instrument driver 62 includes one or more drive units 63 arranged with parallel axes to provide controlled torque to a medical instrument via drive shafts 64. Each drive unit 63 includes an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuity 68 for receiving control signals and actuating the drive unit. Each drive unit 63 being independently controlled and motorized, the instrument driver 62 may provide multiple (four as shown in FIG. 5) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. One purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may include of a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

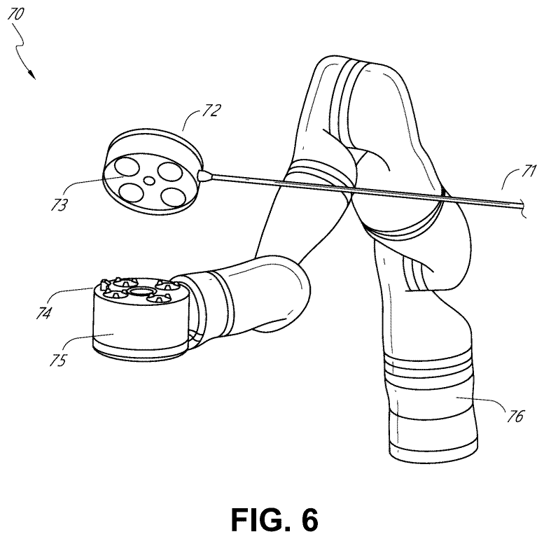

FIG. 6 shows an example medical instrument 70 with a paired instrument driver 75, according to some implementations. Like other instruments designed for use with a robotic system, medical instrument 70 includes an elongated shaft 71 (or elongate body) and an instrument base 72. The instrument base 72, also referred to as an “instrument handle” due to its intended design for manual interaction by the physician, may generally include rotatable drive inputs 73, such as receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of instrument base 72 may share axes of rotation with the drive outputs 74 in the instrument driver 75 to allow the transfer of torque from drive outputs 74 to drive inputs 73. In some implementations, the drive outputs 74 may include splines that are designed to mate with receptacles on the drive inputs 73.

The elongated shaft 71 is designed to be delivered through either an anatomical opening or lumen (such as in endoscopy) or a minimally invasive incision (such as in laparoscopy). The elongated shaft 66 may be either flexible (such as having properties similar to an endoscope) or rigid (such as having properties similar to a laparoscope) or contain a customized combination of both flexible and rigid portions. When designed for laparoscopy, the distal end of a rigid elongated shaft may be connected to an end effector comprising a jointed wrist formed from a clevis with an axis of rotation and a surgical tool, such as, for example, a grasper or scissors, that may be actuated based on force from the tendons as the drive inputs rotate in response to torque received from the drive outputs 74 of the instrument driver 75.

When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons within the shaft 71. These individual tendons, such as pull wires, may be individually anchored to individual drive inputs 73 within the instrument handle 72. From the handle 72, the tendons are directed down one or more pull lumens within the elongated shaft 71 and anchored at the distal portion of the elongated shaft 71. In laparoscopy, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In laparoscopy, the tendon may cause a joint to rotate about an axis, thereby causing the end effector to move in one direction or another. Alternatively, the tendon may be connected to one or more jaws of a grasper at distal end of the elongated shaft 71, where tension from the tendon cause the grasper to close.

In endoscopy, the tendons may be coupled to a bending or articulating section positioned along the elongated shaft 71 (such as at the distal end) via adhesive, control ring, or other mechanical fixation. When fixedly attached to the distal end of a bending section, torque exerted on drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing therebetween may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft may comprise a working channel for deploying surgical tools, irrigation, and/or aspiration to the operative region at the distal end of the shaft 71. The shaft 71 may also accommodate wires and/or optical fibers to transfer signals to or from an optical assembly at the distal tip, which may include of an optical camera. The shaft 71 may also accommodate optical fibers to carry light from proximally-located light sources, such as light emitting diodes, to the distal end of the shaft.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 6, the drive shaft axes, and thus the drive input axes, are orthogonal to the axis of the elongated shaft. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongate shaft 71. The resulting entanglement of such tendons may disrupt any control algorithms intended to predict movement of the flexible elongate shaft during an endoscopic procedure.

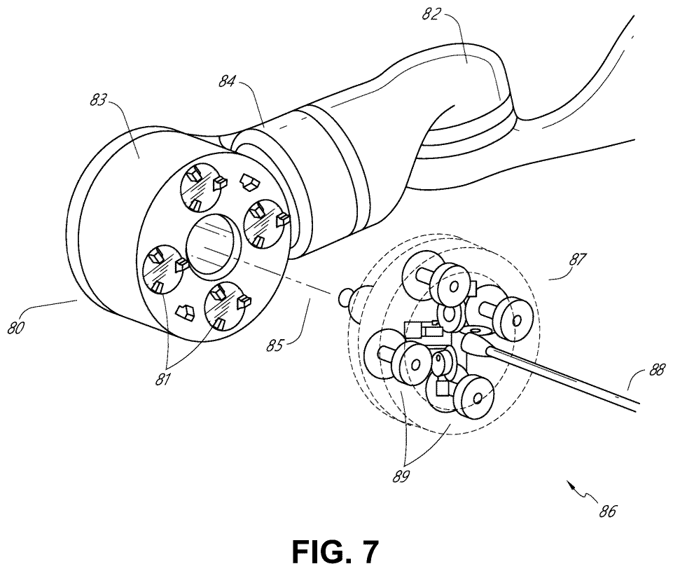

FIG. 7 shows another example medical instrument 86 with a paired instrument driver 80, according to some implementations. As shown, a circular instrument driver 80 includes four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver. Power and controls signals may be communicated from the non-rotational portion 84 of the instrument driver 80 to the rotational assembly 83 through electrical contacts may be maintained through rotation by a brushed slip ring connection (not shown). In some other implementations, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

The instrument 86 may include an elongated shaft portion 88 and an instrument base 87 (shown with a transparent external skin for discussion purposes) having a plurality of drive inputs 89 (such as receptacles, pulleys, and spools) that are configured to receive the drive outputs 81 in the instrument driver 80. Unlike prior disclosed implementations, instrument shaft 88 extends from the center of instrument base 87 with an axis substantially parallel to the axes of the drive inputs 89, rather than orthogonal as in the design of FIG. 6.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

Traditional endoscopy may involve the use of fluoroscopy (such as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

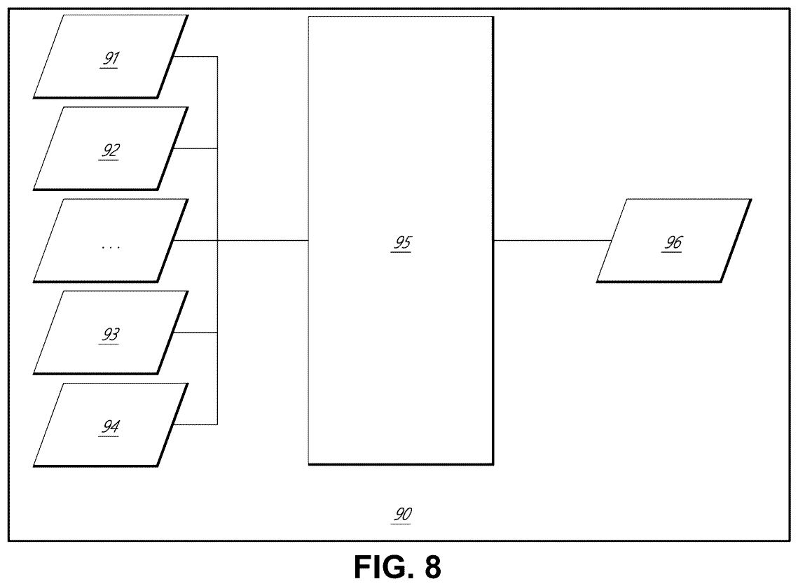

FIG. 8 shows a block diagram of an example localization system 90, according to some implementations. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1 and/or the cart 11 shown in FIG. 1-3.

As shown in FIG. 8, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Pre-operative mapping may be accomplished through the use of the collection of low dose CT scans. Pre-operative CT scans generate two-dimensional images, each representing a “slice” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as preoperative model data 91. Network topological models may also be derived from the CT-images, and are particularly appropriate for bronchoscopy.

In some implementations, the instrument may be equipped with a camera to provide vision data 92. The localization module 95 may process the vision data to enable one or more vision-based location tracking. For example, the preoperative model data may be used in conjunction with the vision data 92 to enable computer vision-based tracking of the medical instrument (such as an endoscope or an instrument advance through a working channel of the endoscope). For example, using the preoperative model data 91, the robotic system may generate a library of expected endoscopic images from the model based on the expected path of travel of the endoscope, each image linked to a location within the model. Intra-operatively, this library may be referenced by the robotic system in order to compare real-time images captured at the camera (such as a camera at a distal end of the endoscope) to those in the image library to assist localization.

Other computer vision-based tracking techniques use feature tracking to determine motion of the camera, and thus the endoscope. Some feature of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising of one or more sensor coils embedded in one or more locations and orientations in a medical instrument (such as an endoscopic tool) measures the variation in the EM field created by one or more static EM field generators positioned at a known location. The location information detected by the EM sensors is stored as EM data 93. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively “registered” to the patient anatomy (such as the preoperative model) in order to determine the geometric transformation that aligns a single location in the coordinate system with a position in the pre-operative model of the patient's anatomy. Once registered, an embedded EM tracker in one or more positions of the medical instrument (such as the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during pre-operative calibration. Intra-operatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 8 shows, a number of other input data can be used by the localization module 95. For example, although not shown in FIG. 8, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94.

Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

The robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system or anatomical map.

As described above, some medical instruments (such as an access sheath) are designed to be used in close proximity and coordination with other medical instruments (such as a scope or catheter) such that relative misalignments between the robotic arms that manipulate the instruments can cause the medical instruments to make unwanted contact or otherwise exert undesired forces against one another. For example, during a bronchoscopy, an endoscope can be inserted and retracted within a lumen of an access sheath so that the instruments are coaxial with one another while being driven by respective robotic arms. To preserve the coaxiality of such instruments, the end effectors and/or IDMs of the robotic arms may be configured to move along a “virtual rail.” However, because each instrument is coupled to and manipulated by a respective robotic arm, relative misalignment between the arms can result in loss of coaxiality between the instruments thereby causing the outer surface of the endoscope to press or rub against the inner surface of the access sheath as the instruments are driven in relation to one another. Such forces can cause damage to the instruments and/or overexertion of various motors or actuators in the robotic arms.

The relative misalignment between robotic arms also causes the instruments to exert torque or other counterforces against the motors or actuators in the robotic arms. Aspects of the present disclosure recognize that such forces can be detected and/or measured by the motors themselves, or by various sensors in a robotic arm, and further used to guide realignment of the robotic arms. For example, each IDM may include one or more torque sensors that are configured to measure torque on the IDM. The measured torque (τ) is equal to the sum of any torque (τ_{D}) exerted by the drive motor(s) internal to the IDM (also referred to as “drive torque”) and any counter-torque (τ_{M}) exerted by the instrument coupled to the IDM (also referred to as “misalignment torque”). More specifically, the misalignment torque τ_{M} can be determined as a function of the measured torque τ and the drive torque τ_{D}, where τ_{M}=τ−τ_{D}. In some aspects, a robotic system may determine whether the misalignment torque τ_{M} is outside a normal or expected range (associated with driving instruments that are coaxially aligned) so as to indicate relative misalignment between the robotic arms.

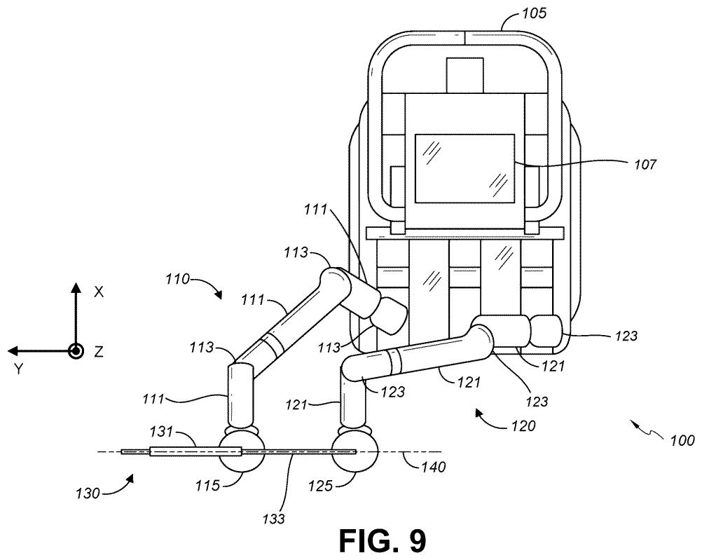

FIG. 9 shows another example robotic system 100, according to some implementations. In some implementations, the robotic system 100 may be one example of any of the robotic systems shown in FIG. 1-4. The robotic system 100 includes a cart 105 having a number of robotic arms 110 and 120 coupled to a steerable instrument 130. With reference to FIG. 2, the cart 105 may be one example of the cart 11 and each of the robotic arms 110 and 120 may be one example of any of the robotic arms 12. Although only two robotic arms 110 and 120 are shown in the example of FIG. 9, the robotic system 100 may include three or more robotic arms in some other implementations (such as shown in FIG. 1-4).

In some implementations, the cart 105 may include a processor and a memory (not shown for simplicity) that store and execute instructions for controlling and/or manipulating the robotic arms 110 and 120. In the example of FIG. 9, the cart 105 is also shown to include a display 107 which may provide a user interface for operating the robotic system 100. In some other implementations, one or more of the processor, the memory, or the display 107 may be located on or within another device (such as the moveable tower 30 of FIG. 1). Still further, in some implementations, a feedback device (different than the display 107) may be provided in lieu of, or in addition to, the display 107. Example suitable feedback devices include haptic devices, speakers, force-feedback actuated via one or more of the robotic arms 110 or 120, and light-emitting diodes (LEDs), among other examples.

The first robotic arm 110 includes a number of linkages 111, a number of joints 113, and an IDM 115 coupled to the steerable instrument 130. Each of the joints 113 connects two adjacent linkages 111. In some implementations, the first robotic arm 110 also may include one or more torque sensors (not shown for simplicity) configured to detect torque between two of the linkages 111 and/or the IDM 115. For example, a given joint 113 may include a corresponding torque sensor configured to detect torque between the two linkages 111 adjacent to that joint 113. Thus, the torque sensor may be located in the joint 113 and may be coupled to the two adjacent linkages 111. A torque sensor also may be provided in the joint 113 that connects the first robotic arm 110 to the cart 105. In some implementations, the torque sensor(s) may be implemented as a plurality of strain gauges configured to reduce or minimize the sensing of any torque that is not along the axis of rotation of the corresponding joint 113.

In some implementations, each of the joints 113 may include a motor (not shown for simplicity) that is coupled to the two adjacent linkages 111. Thus, a given joint 113 may further house the motor, which is configured to apply a force between the two adjacent linkages 111 (or between a linkage 111 and the cart 105) in order to adjust the positioning of the two adjacent linkages 111. As shown in FIG. 9, the IDM 115 is coupled to a distal end of the robotic arm 110. By actuating the motor in one or more of the joints 113 of the first robotic arm 110, the motors may be operable to adjust the posture or pose of the first robotic arm 110, and thus the IDM 115 (such as by adjusting the position and/or orientation of one or more joints 113 of the first robotic arm 110), and thereby control the steerable instrument coupled to the IDM 115.

In some implementations, each of the joints 113 may further include a position sensor configured to measure the relative position of the two adjacent linkages 111. The position sensor of a given joint 113 may be configured to measure the angle between the two adjacent linkages 111. The system may determine the position of each of the linkages 111 in the first robotic arm 110 based on information provided by each of the position sensors. In some implementations, a position sensor may include an encoder configured to measure the speed and/or position of the motor shaft by reading, for example, coded visual information printed on the motor shaft and may provide feedback to the system representative of the speed and/or position of the motor.

Similar to the first robotic arm 110, the second robotic arm 120 may include a number of linkages 121, a number of joints 123 connecting adjacent linkages 121, and an IDM 125. Each of the joints 123 may further include a corresponding torque sensor, motor, and/or position sensor (not shown for simplicity). The IDM 125 also may be coupled to the steerable instrument 130 to drive or otherwise manipulate the instrument.

In some implementations, the motors themselves may measure the torque on the joints 113 and 123 (such as in lieu of a torque sensor). For example, when a force is applied to the first robotic arm 110 (such as the force of gravity, the force of a collision, or a force exerted by a user), the motor(s) may be configured to apply opposite and opposing force(s) to the joints 113 to maintain the pose of the first robotic arm 110. The current in the motor(s) required to maintain the pose of the first robotic arm 110, or otherwise resist such external forces, may represent the torque that is applied to the corresponding joints 113.

The steerable instrument 130 comprises a first medical instrument 131 (such as an access sheath) coupled to the IDM 115 of the first robotic arm 110 and a second medical instrument 133 (such as a scope or catheter) coupled to the IDM 125 of the second robotic arm 120. The medical instruments 131 and 133 may be configured to be advanced and/or inserted into (and retracted from) a patient or an anatomy along an axis 140 (also referred to as the “virtual rail”). For example, the virtual rail 140 may be defined as the axis of alignment of the IDMs 115 and 125. Movement of the IDMs 115 and 125 along the virtual rail 140 may control the advancing and retracting of the medical instruments 131 and 133. The poses (including the positions and/or orientations) of the IDMs 115 and 125 may be described with respect to a 3D coordinate space (X, Y, Z). As shown in FIG. 9, the Y-axis is parallel to the virtual rail 140, the Z-axis is parallel to the direction of gravity, and the X-axis is perpendicular to the Y-Z plane. However, the coordinate space may be defined differently in some other implementations.

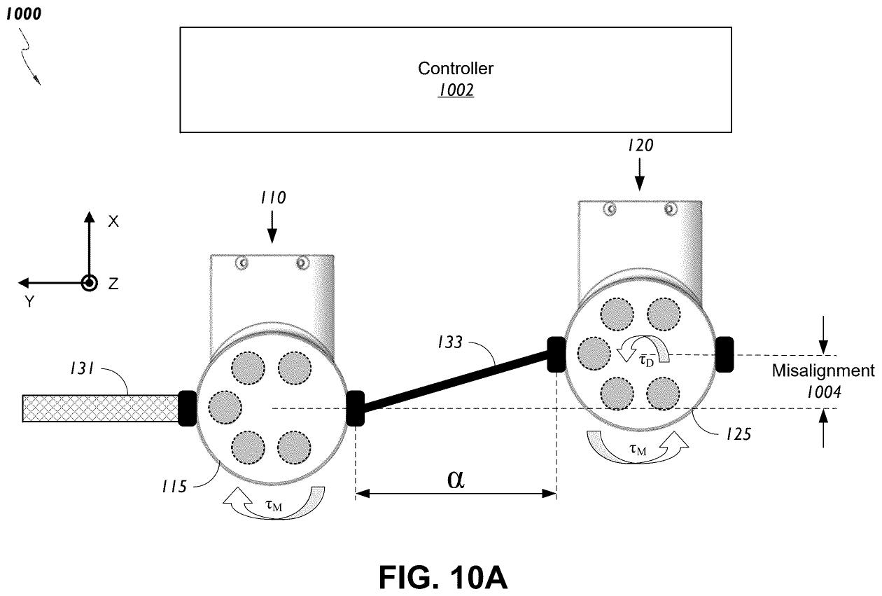

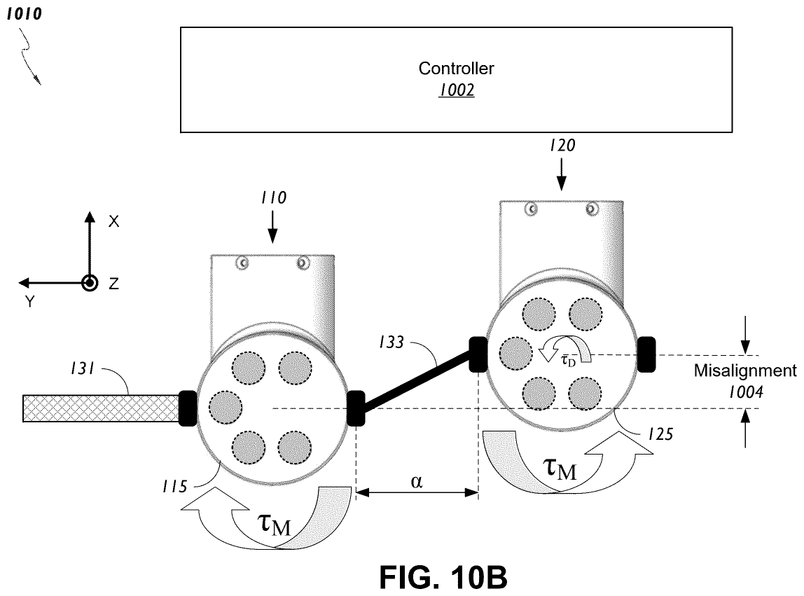

FIG. 10A shows an example configuration 1000 of the robotic system 100 of FIG. 9 in which the robotic arms 110 and 120 are misaligned. FIG. 10A also shows a controller 1002 for the robotic system 100. In some aspects, the controller 1002 may be implemented by processing and memory resources within the cart 105. In some other aspects, the controller 1002 may be implemented by processing and memory resources in other parts of a medical system (such as the tower 30 of FIG. 1).

The controller 1002 is configured to align the robotic arms 110 and 120 along the virtual rail 140 based on a kinematic model. However, due to inaccuracies in the kinematic model (which may be attributable to process variations in manufacturing, mounting inaccuracies, and/or temperature changes) the robotic arms 110 and 120 are misaligned 1004 such that the first robotic arm 110 may not move along the same virtual rail (in the Y-direction) as the second robotic arm 120. As shown in FIG. 10A, the second robotic arm 120 has a greater offset in the X-direction (and thus closer to the controller 1002) than the first robotic arm 110. For simplicity, the misalignment 1004 is shown only in the X-direction. However, in actual implementations, the robotic arms 110 and 120 and/or IDMs 115 and 125 may be misaligned in position and/or orientation with various degrees of freedom.

Due to the relative misalignment 1004, the instruments 131 and 133 are not coaxial (in the Y-direction) and may exert unwanted forces against one another. For example, the outer surface of the second instrument 133 may press or rub against the inner surface of the first instrument 131. Such unwanted forces may be transmitted to the IDMs 115 and 125 in the form of excess torque (τ_{M}) or resistance to any drive torque (τ_{D}) exerted by the motors in the IDMs 115 and 125. Aspects of the present disclosure recognize that the excess torque τ_{M} (also referred to as the “misalignment torque”) becomes greater as the distance (α) between the IDMs 115 and 125, and/or robotic arms 110 and 120, becomes shorter (such as when driving of the second instrument 133 inside the lumen of the first instrument 131).

FIG. 10B shows another example configuration 1010 of the robotic system 100 of FIG. 9 in which the robotic arms 110 and 120 are misaligned. Although the misalignment 1004 between the IDMs 115 and 125 (in the X-direction) is the same between both configurations 1000 and 1010, the distance α between the robotic arms 110 and 120 and the IDMs 115 and 125 is much shorter in the configuration 1010 of FIG. 10B compared to the configuration 1000 of FIG. 10A. As a result, the misalignment torque τ_{M} on the IDMs 115 and 125 is shown to be substantially greater in the configuration 1010 of FIG. 10B compared to the configuration 1000 of FIG. 10A. More generally, the misalignment torque τ_{M} is proportional to the degree of misalignment 1004 and inversely proportional to the distance α between the robotic arms 110 and 120 (and specifically, the IDMs 115 and 125).

In the example of FIGS. 10A and 10B, the first robotic arm 110 is shown to remain stationary while the second robotic arm 120 is driven closer to the first robotic arm 110. However, in some other implementations, the first robotic arm 110 may move concurrently or separately from the second robotic arm 120. For example, when inserting the instruments 131 and 133 into a patient or anatomy, the robotic arms 110 and 120 may concurrently drive the instruments 131 and 133 along a virtual rail until a threshold insertion depth is reached. The second robotic arm 120 may then drive the second instrument 133 further into the anatomy, beyond the threshold insertion depth, while the first robotic arm 110 holds the first instrument 131 stationary. When the procedure is completed, the second robotic arm 120 may retract the second instrument 133 back to the threshold insertion depth, at which point the robotic arms 110 and 120 may concurrently retract the instruments 131 and 133 out along the virtual rail.

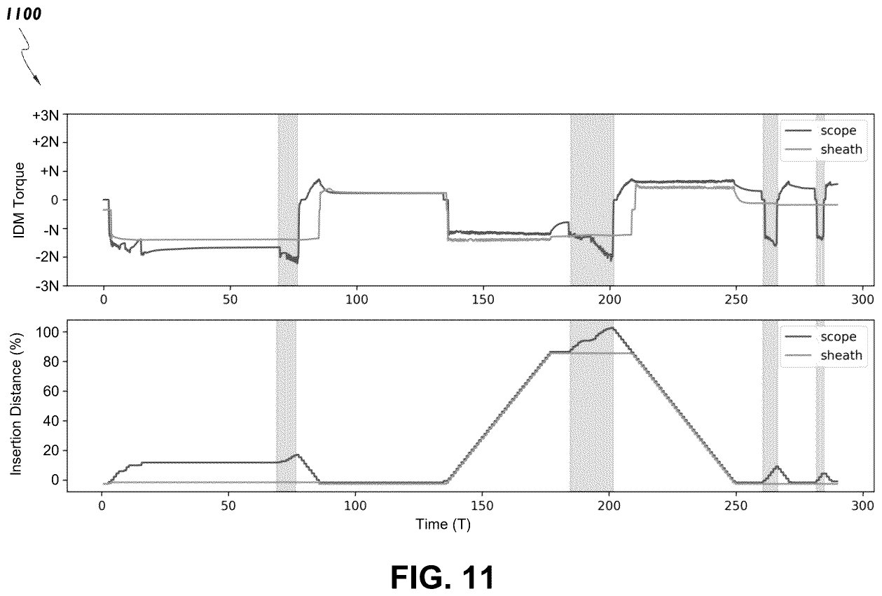

FIG. 11 shows a timing diagram 1100 depicting an example insertion and retraction of medical instruments coupled to respective robotic arms. In the example of FIG. 11, the medical instruments are shown to include a scope and a sheath. In some implementations, the sheath and the scope may be examples of the first instrument 131 and the second instrument 133, respectively, of FIGS. 10A and 10B. With reference for example to FIGS. 10A and 10B, the sheath may be coupled to the first robotic arm 110 and the scope may be coupled to the second robotic arm 120.

In the example of FIG. 11, IDM torque and insertion distance are described in relative terms, which may reflect any real-world values that can be recreated during insertion and retraction of a medical instrument. For example, IDM torque equal to “0” represents the resting torque of an IDM (without an instrument-driving forces being exerted or applied), which may correspond to a value greater or less than 0 Nm in actual procedures. Similarly, a 0% insertion distance reflects the position of the instruments at the start of the insertion/retraction operation depicted in FIG. 11, which may correspond to a value greater than 0 mm in actual procedures. Accordingly, the values depicted in FIG. 11 should not be construed as limiting the scope of this disclosure to any particular values and/or ranges of values.

In the example of FIG. 11, the scope is disposed within a lumen of the sheath while the robotic arms are relatively misaligned according to a kinematic model (such as described with reference to FIGS. 10A and 10B). As a result, the scope is not coaxial with the sheath. At time T=0, both the scope and the sheath are initially positioned at 0% insertion depth. Between times T=0 and T=135, the sheath remains stationary while the scope is inserted to an insertion depth≈20% (between times T=0 and T=80) and retracted back to 0% insertion depth (between times T=80 and T=135). Due to the relative misalignment of the robotic arms, the insertion and retraction of the scope exerts misalignment torque on the IDMs coupled to the scope and the sheath.

As shown in FIG. 11, the IDM coupled to the sheath registers a torque≈−N (where N represents any real-world torque value) between times T=0 and T=90 while its insertion depth does not change. This torque may be exerted by a motor in the IDM to resist the misalignment torque while maintaining the current pose of the sheath. FIG. 11 also shows a downward slope in the torque curve for the IDM coupled to the scope between times T=70 and T=80 (highlighted by gray bars). This may represent the excess torque that needs to be exerted by a motor in the IDM to overcome the increasing misalignment torque as the distance between the IDMs is reduced (such as described with reference to FIGS. 10A and 10B). As described with reference to FIG. 9, the torque on the IDMs may be sensed or measured by various means (including torque or force sensors in the IDMs and/or feedback from the motors themselves).

Between times T=135 and T=175, the scope and the sheath are concurrently driven to an insertion depth≈90%. Then, between times T=175 and T=200, the scope is driven further to an insertion depth≈100% while the sheath remains stationary. FIG. 11 again shows a downward slope in the torque curve for the IDM coupled to the scope (highlighted by gray bars), between times T=175 and T=200, while driving to the maximum insertion depth. As described above, this may represent the excess torque that needs to be exerted by a motor in the IDM to overcome the increasing misalignment torque as the distance between the IDMs is reduced. In the example of FIG. 11, the scope is retracted back to an insertion depth≈90% between times T=200 and T=210.

Between times T=210 and T=250, the sheath and the scope are concurrently retracted back to 0% insertion depth. In the example of FIG. 11, the scope is subsequently inserted (to an insertion depth≈5-10%) and retracted, twice, while the sheath remains stationary between times T=250 and T=300. During each of these movements, the IDM coupled to the scope registers an increasing amount of torque (highlighted by gray bars) as the motor(s) in the IDM attempt to overcome the increasing misalignment torque in order to reach the target instrument pose.

Aspects of the present disclosure recognize that the degree and magnitude of the misalignment torque can guide the selection of a kinematic offset to be applied to one or more robotic arms to correct the misalignment between the arms. For example, the robotic system may iteratively adjust a kinematic model so that at least one of the robotic arms has a new position and/or orientation when operated in relation to the other robotic arm (such as along a virtual rail). For each iteration, the robotic system may determine whether the adjustments to the kinematic model result in lowering the misalignment torque. As such, the robotic system may iteratively adjust the kinematic model until the misalignment torque is minimized and/or falls within a threshold range.

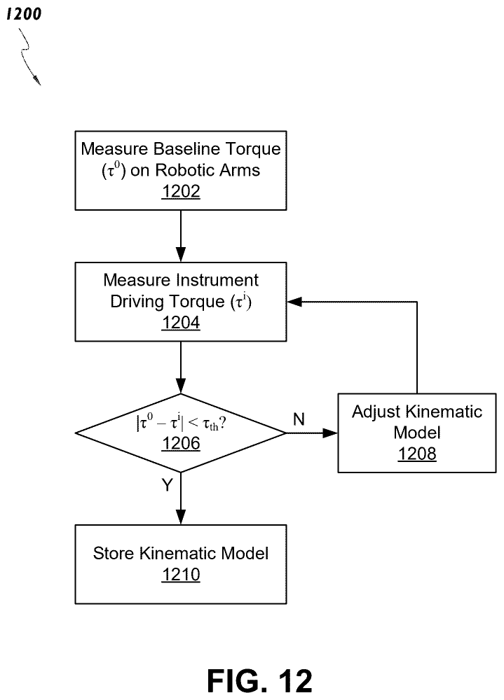

FIG. 12 shows an illustrative flowchart depicting an example process 1200 for iteratively adjusting a kinematic model to compensate for relative misalignment of robotic arms. In some implementations, the example operation 1200 may be performed by a robotic system having multiple robotic arms, such as the robotic system 100 of FIG. 9 or any of the robotic systems shown in FIG. 1-4.

The process 1200 begins, at 1202, with the robotic system measuring a baseline torque (τ⁰) on each of the robotic arms. The baseline torque τ⁰ on a given robotic arm represents the torque exerted by one or more motors of the robotic arm to move one or more joints based on a kinematic model (and overcome any inherent friction or “stickiness” in the joints) without any medical instruments. In some aspects, a controller for the robotic system (such as the controller 1002 of FIGS. 10A and 10B) may measure the baseline torque τ⁰ on a robotic arm based on feedback provided by sensors and/or motors in the robotic arm while manipulating the arm through various poses associated with a medical procedure (such as to move an IDM along a virtual rail) without any medical instruments coupled thereto. In some implementations, the controller may measure a baseline torque τ⁰ at a single location (such as a joint or IDM) in each robotic arm. In some other implementations, the controller may measure baseline torques τ⁰ at multiple locations in each robotic arm.

At 1204, the robotic system measures an instrument driving torque (τⁱ) on each of the robotic arms. The instrument driving torque τⁱ on a given robotic arm represents the torque exerted by one or more motors of the robotic arm to drive a medical instrument based on the kinematic model. In some aspects, the controller for the robotic arms may measure the instrument driving torque τⁱ on a robotic arm based on feedback provided by sensors and/or motors in the robotic arm while manipulating the arm through various poses that cause a first medical instrument coupled to the arm to be driven in relation to a second medical instrument coupled to another robotic arm (such as by performing an insertion and/or retraction of a scope or catheter within the lumen of a sheath). More specifically, the arm poses used to measure the baseline torque τ⁰ may mirror the arm poses used to measure the instrument driving torque τⁱ. In some implementations, the controller may measure an instrument driving torque τⁱ at a single location (such as a joint or IDM) in each robotic arm. In some other implementations, the controller may measure instrument driving torques τⁱ at multiple locations in each robotic arm.

At 1206, the robotic system compares the instrument driving torque τⁱ with the baseline torque τ⁰ to determine whether the robotic arms are relatively misaligned. Aspects of the present disclosure recognize that the motors of the robotic arms may need to exert more torque than the baseline torque τ⁰ to drive a set of medical instruments in accordance with a medical procedure (such as to drive a scope or catheter inside the lumen of a sheath) even when the arms are relatively aligned. Thus, in some implementations, the controller for the robotic arms may determine that the robotic arms are relatively misaligned only if the instrument driving torque τⁱ for at least one of the robotic arms differs from the baseline torque τ⁰ for the robotic arm by a threshold amount (τₜₕ). If the robotic system determines that the robotic arms are relatively aligned (such as where |τ⁰−τⁱ|<τₜₕ), at 1206, the robotic system may proceed to 1210 by storing the current kinematic model. Accordingly, the robotic system may subsequently use the stored kinematic model to perform the medical procedure.

Otherwise, if the robotic system determines that the robotic arms are relatively misaligned (such as where |τ⁰−τⁱ|≥τₜₕ), at 1206, the robotic system may proceed to 1208 by adjusting the kinematic model. More specifically, the controller for the robotic arms may adjust the kinematic model so that at least one of the robotic arms is posed differently relative to the other robotic arm(s) when performing the medical procedure. In some implementations, the controller may adjust a kinematic offset to be applied to one or more of the robotic arms. For example, the kinematic offset may alter the position and/or orientation of a robotic arm when aligned with a virtual rail. In some aspects, the controller may determine the adjustment to the kinematic model based on the magnitude and/or direction of a misalignment torque (τ_{M}), where τ_{M}=τ⁰−τⁱ. For example, if the misalignment torque τ_{M} indicates that an instrument coupled to an IDM is transmitting forces to the IDM in a positive X-direction, the controller may apply a kinematic offset that moves the IDM in a negative X-direction when aligned with the virtual rail. Alternatively, or additionally, the controller may apply a kinematic offset that moves the IDM coupled to the other instrument in a positive X-direction when aligned with the virtual rail.

After adjusting the kinematic model, at 1208, the robotic system proceeds to 1204 by measuring the instrument driving torque τⁱ based on the adjusted kinematic model. Thus, by repeating steps 1204-1208, the controller for the robotic system may iteratively adjust the kinematic model until the instrument driving torque τⁱ for the i^{th} iteration is within a threshold deviation of the baseline torque τ⁰ (where |τ⁰−τⁱ|<τₜₕ). In some aspects, the example process 1200 of FIG. 12 may be repeated with the robotic arms repositioned to perform the medical procedure from a different direction. With reference for example to FIG. 1, the cart 11 may be positioned on either side (such as to the left or the right) of a patient when the robotic system is deployed in the field. Thus, the controller for the robotic system may determine kinematic offsets for multiple arm directions (such as where the cart 11 is positioned to the left of a patient and where the cart 11 is positioned to the right of a patient) to ensure proper alignment of the robotic arms regardless of how the robotic system is deployed. In some implementations, the controller may implement an average of the kinematic offsets between the different directions.

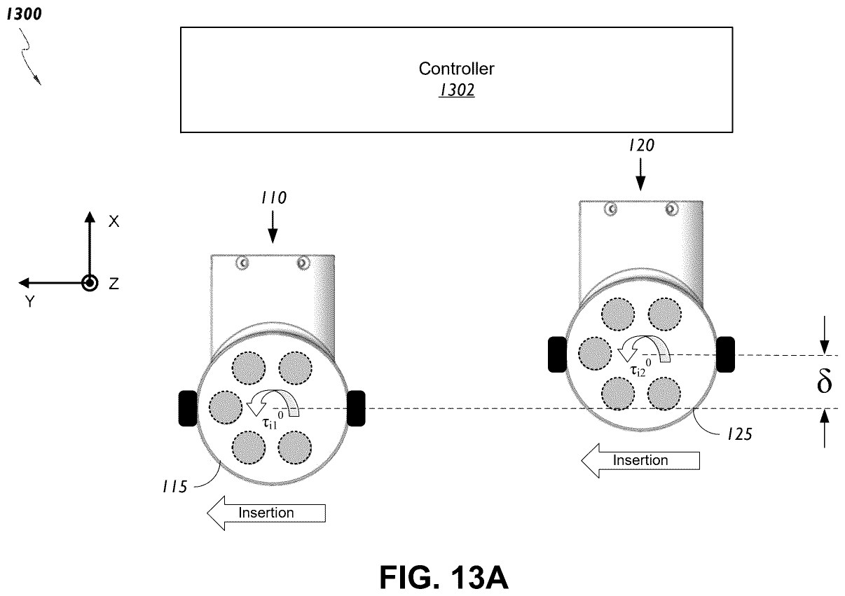

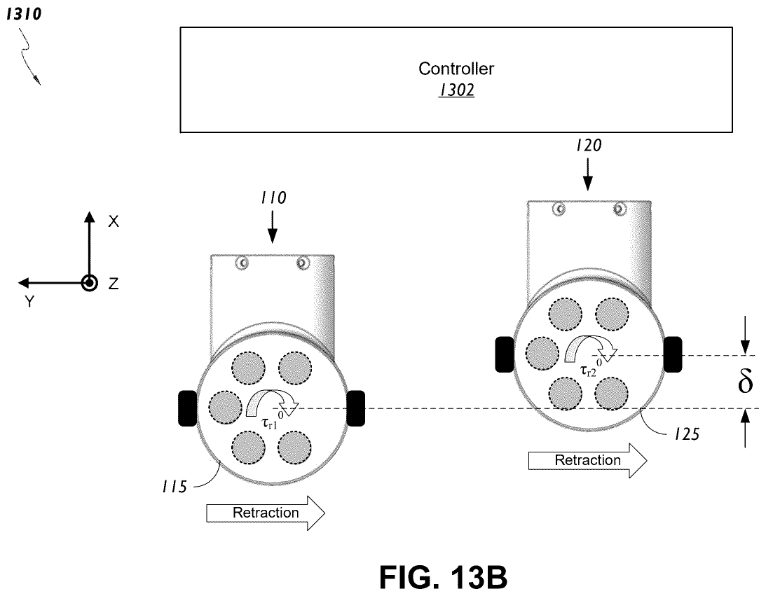

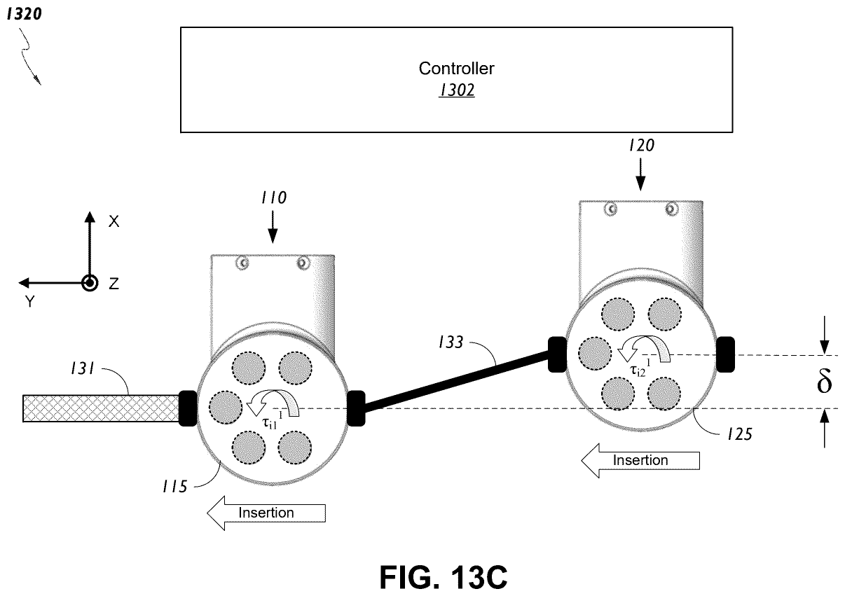

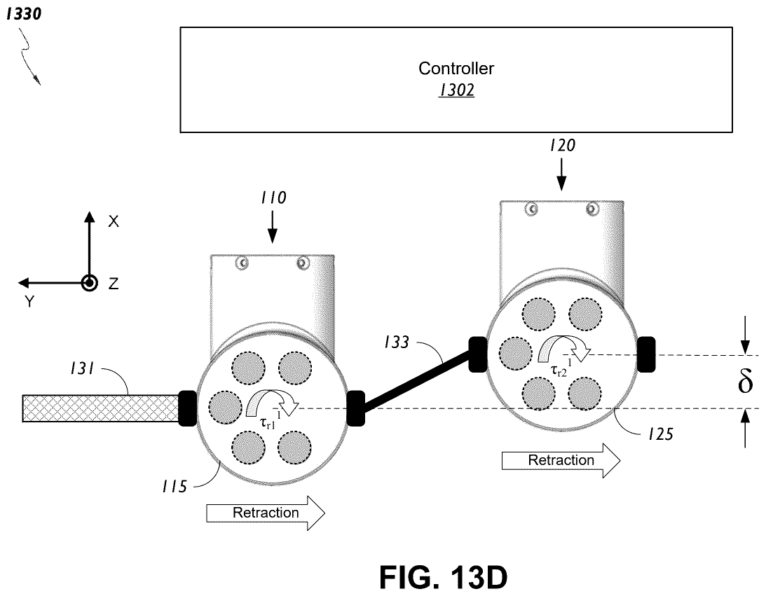

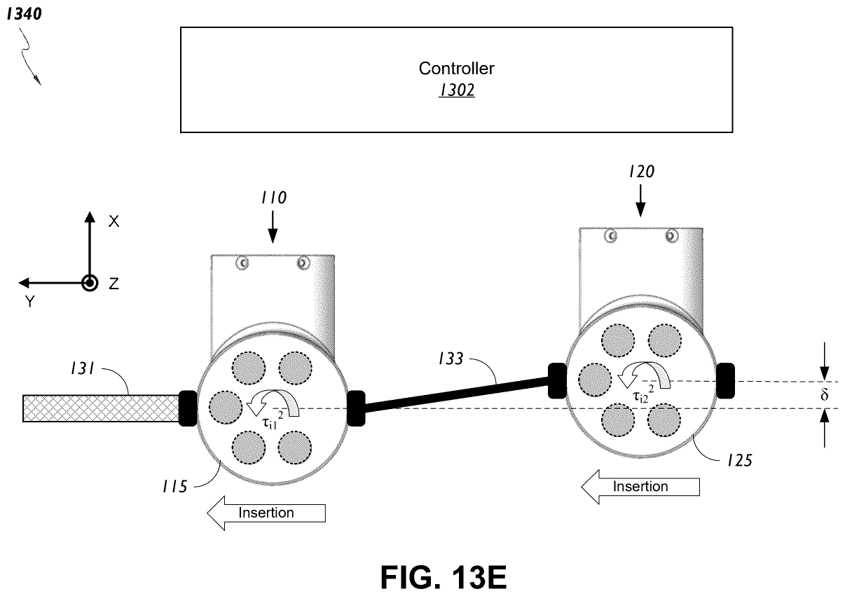

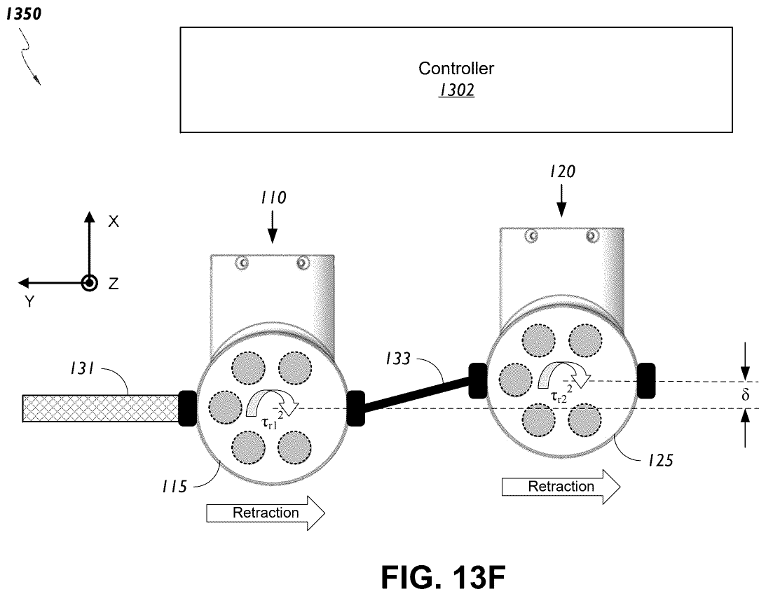

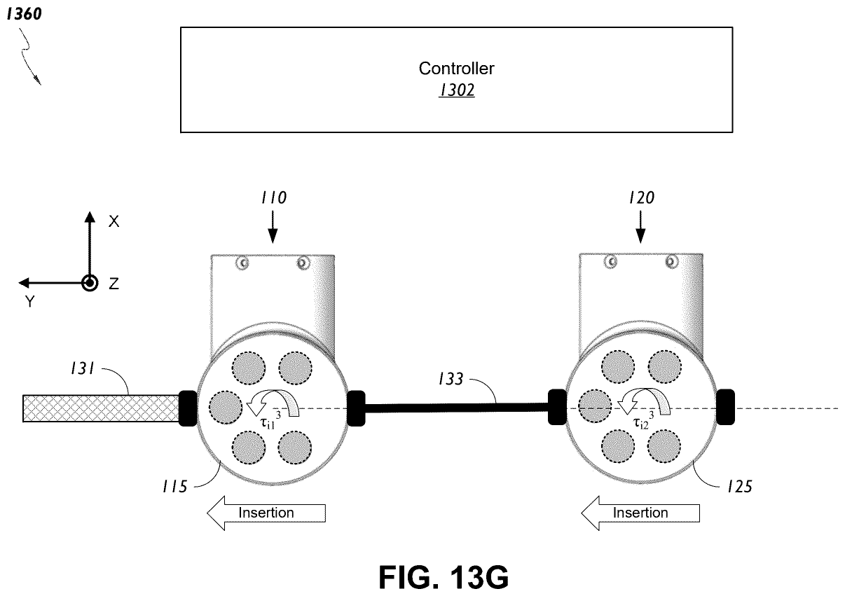

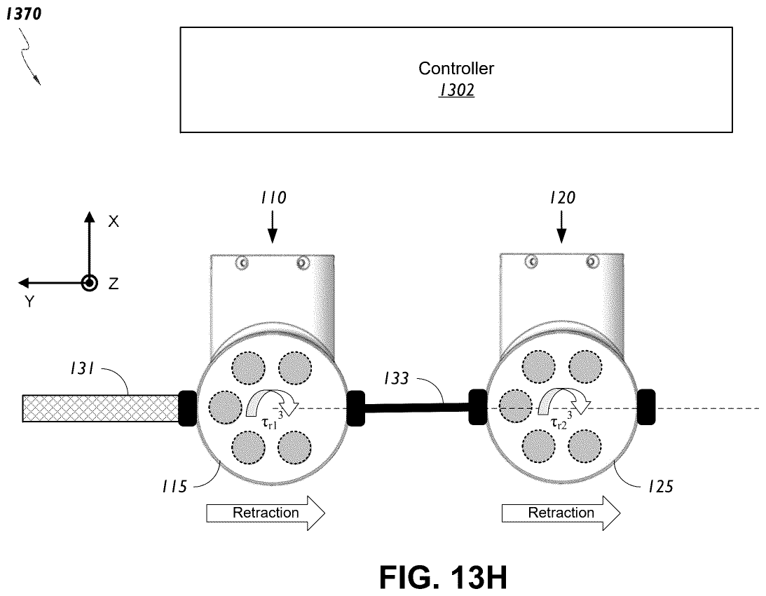

FIG. 13A-13H show example configurations 1300-1370, respectively, of the robotic system 100 of FIG. 9 for determining and correcting relative misalignment between the robotic arms 110 and 120. More specifically, the configurations 1300-1370 depict example movements of the robotic arms 110 and 120 for performing the process 1200 of FIG. 12. For example, FIGS. 13A and 13B show example robotic arm movements for measuring a baseline torque τ⁰, FIGS. 13C and 13D show example robotic movements for measuring an instrument driving torque τ¹ according to a first iteration of the process 1200, FIGS. 13E and 13F show example robotic arm movements for measuring an instrument driving torque τ² according to a second iteration of the process 1200, and FIGS. 13G and 13H show example robotic arm movements for measuring an instrument driving torque τ³ according to a third iteration of the process 1200. In the example of FIG. 13A-13H, the movements of the robotic arms 110 and 120 are controlled by a controller 1302 for the robotic system 100. In some implementations, the controller 1302 may be one example of the controller 1002 of FIGS. 10A and 10B.

With reference to FIG. 13A, the controller 1302 moves the robotic arms 110 and 120 through a series of poses that simulate an insertion of a medical instrument (such as into a patient or anatomy) but without any instruments coupled to the IDMs 115 and 125. For example, the controller 1302 may control various motors of the robotic arms 110 and 120 to move the IDMs 115 and 125 along a virtual rail, in the positive Y-direction, based on a kinematic model. In some implementations, the controller 1302 may measure baseline torques τᵢ₁⁰ and τᵢ₂⁰ on the IDMs 115 and 125, respectively, or on motors that drive the IDMs during the simulation of the instrument insertion. The controller 1302 may measure the baseline torques τᵢ₁⁰ and τᵢ₂⁰ using any suitable means (such as described with reference to FIGS. 9 and 12).

With reference to FIG. 13B, the controller 1302 moves the robotic arms 110 and 120 through a series of poses that simulate a retraction of a medical instrument (such as from a patient or anatomy) but without any instruments coupled to the IDMs 115 and 125. For example, the controller 1302 may control various motors of the robotic arms 110 and 120 to move the IDMs 115 and 125 along a virtual rail, in the negative Y-direction, based on the kinematic model. In some implementations, the controller 1302 may measure baseline torques τᵣ₁⁰ and τᵣ₂⁰ on the IDMs 115 and 125, respectively, or on motors that drive the IDMs during the simulation of the instrument retraction. The controller 1302 may measure the baseline torques τᵣ₁⁰ and τᵣ₂ ⁰ using any suitable means (such as described with reference to FIGS. 9 and 12).

In the example of FIGS. 13A and 13B, the kinematic model indicates that the IDMs 110 and 120 are aligned along the same axis in the Y-direction when locked to the virtual rail (such as the virtual rail 140 of FIG. 9). However, as shown in FIG. 13A, inaccuracies in the kinematic model result in a relative misalignment (δ) between the IDMs 110 and 120 when the controller 1302 attempts to align each IDM to the virtual rail. For simplicity, the relative misalignment δ is shown to be only in the X-direction in FIGS. 13A and 13B. However, in actual implementations, the robotic arms 110 and 120 and/or IDMs 115 and 125 may be misaligned in position and/or orientation with various degrees of freedom. Thus, in some other aspects, the controller 1302 may measure torques on various other joints and/or motors of the robotic system 100 to detect relative misalignments in other dimensions.

With reference to FIG. 13C, the controller 1302 moves the robotic arms 110 and 120 through a series of poses that simulate the insertion of a medical instrument (such as into a patient or anatomy) while the instruments 131 and 133 are coupled to the IDMs 115 and 125, respectively. For example, the controller 1302 may control various motors of the robotic arms 110 and 120 to move the IDMs 115 and 125 through the same series of poses used to measure the baseline torques τᵢ₁⁰ and τᵢ₁⁰ (such as in FIG. 13A) based on the kinematic model. In some implementations, the controller 1302 may measure instrument driving torques τᵢ₁¹ and τᵢ₁¹ on the IDMs 115 and 125, respectively, or on motors that drive the IDMs during the simulation of the instrument insertion. The controller 1302 may measure the instrument driving torques τᵢ₁¹ and τᵢ₁¹ using any suitable means (such as described with reference to FIGS. 9 and 12).

With reference to FIG. 13D, the controller 1302 moves the robotic arms 110 and 120 through a series of poses that simulate the retraction of a medical instrument (such as from a patient or anatomy) while the instruments 131 and 133 are coupled to the IDMs 115 and 125, respectively. For example, the controller 1302 may control various motors of the robotic arms 110 and 120 to move the IDMs 115 and 125 through the same series of poses used to measure the baseline torques τᵣ₁⁰ and τᵣ₂⁰ (such as in FIG. 13B) based on the kinematic model. In some implementations, the controller 1302 may measure instrument driving torques τᵣ₁¹ and τᵣ₁¹ on the IDMs 115 and 125, respectively, or on motors that drive the IDMs during the simulation of the instrument retraction. The controller 1302 may measure the instrument driving torques τᵣ₁¹ and τᵣ₂¹ using any suitable means (such as described with reference to FIGS. 9 and 12).

In some implementations, the controller 1302 may compare the instrument driving torques with the baseline torques to determine whether the robotic arms 110 and 120 and/or the IDMs 115 and 125 are relatively misaligned. As described with reference to FIG. 12, the controller 1302 may determine a misalignment torque on each of the IDMs 115 and 125 based on the comparison. For example, the controller 1302 may determine that the IDMs 115 and 125 are relatively misaligned if |τᵢ₁⁰−τᵢ₁¹|≥τₜₕ, if |τᵢ₂⁰−τᵢ₂¹|≥τₜₕ, if |τᵣ₁⁰−τᵣ₁¹|≥Tₜₕ, and/or if |τᵣ₂⁰−τᵣ₂¹|≥τ_{th,} where τₜₕ represents a threshold deviation from the baseline torque on a given IDM. In the example of FIGS. 13C and 13D, the controller 1302 may determine that the IDMs 115 and 125 are relatively misaligned based on comparing the instrument driving torques with the baseline torques for insertion and retraction of a medical instrument. The controller 1302 may further determine, based on the magnitude and direction of the misalignment torques, that the IDM 125 has a positive offset in the X-direction relative to the IDM 115.

In some implementations, the controller 1302 may adjust the kinematic model (such as by applying a kinematic offset to the second robotic arm 120) so that the IDM 125 is pushed further in the negative X-direction when the controller 1302 aligns the IDM 125 to the virtual rail. In some other implementations, the controller 1302 may adjust the kinematic model (such as by applying a kinematic offset to the first robotic arm 110) so that the IDM 115 is pushed further in the positive X-direction when the controller 1302 aligns the IDM 115 to the virtual rail. Still further in some implementations, the controller 1302 may apply kinematic offsets to both robotic arms 110 and 120. As shown in FIGS. 13E and 13F, the adjusted kinematic model moves the IDMs 110 and 120 closer to one another in the X-direction when the controller 1302 aligns each IDM to the virtual rail, thereby reducing the relative misalignment δ between the robotic arms 110 and 120.

With reference to FIG. 13E, the controller 1302 moves the robotic arms 110 and 120 through a series of poses that simulate the insertion of a medical instrument, based on the adjusted kinematic model, while the instruments 131 and 133 are coupled to the IDMs 115 and 125, respectively (such as described with reference to FIG. 13C). The controller 1302 also measures instrument driving torques τᵢ₁² and τᵢ₂² on the IDMs 115 and 125, respectively, or on motors that drive the IDMs during the instrument insertion. With reference to FIG. 13F, the controller 1302 moves the robotic arms 110 and 120 through a series of poses that simulate the retraction of a medical instrument, based on the adjusted kinematic model, while the instruments 131 and 133 are coupled to the IDMs 115 and 125, respectively (such as described with reference to FIG. 13D). The controller 1302 also measures instrument driving torques τᵣ₁² and τᵣ₂² on the IDMs 115 and 125, respectively, or on motors that drive the IDMs during the instrument retraction.

In some implementations, the controller 1302 may compare the instrument driving torques with the baseline torques to determine whether the robotic arms 110 and 120 and/or the IDMs 115 and 125 are still misaligned (such as described with reference to FIG. 12). For example, the controller 1302 may determine that the IDMs 115 and 125 are relatively misaligned if |τᵢ₁⁰−τᵢ₁²|≥τₜₕ, if |τᵢ₂⁰−τᵢ₂²|≥τₜₕ, if |τᵣ₁⁰−τᵣ₁²|≥τₜₕ, and/or if |τᵣ₂⁰−τᵣ₂²|≥τₜₕ. In the example of FIGS. 13E and 13F, the controller 1302 may determine that the IDMs 115 and 125 are still misaligned based on comparing the instrument driving torques with the baseline torques for insertion and retraction of a medical instrument. The controller 1302 may further determine, based on the magnitude and direction of the misalignment torques, that the IDM 125 is still offset relative to the IDM 115 in the positive X-direction.

In some implementations, the controller 1302 may further adjust the kinematic model (such as by adjusting the kinematic offset to the second robotic arm 120) so that the IDM 125 is pushed further in the negative X-direction when the controller 1302 aligns the IDM 125 to the virtual rail. In some other implementations, the controller 1302 may further adjust the kinematic model (such as by adjusting the kinematic offset to the first robotic arm 110) so that the IDM 115 is pushed further in the positive X-direction when the controller 1302 aligns the IDM 115 to the virtual rail. Still further in some implementations, the controller 1302 may apply kinematic offsets to both robotic arms 110 and 120. As shown in FIGS. 13G and 13H, as a result of further adjusting the kinematic model, the IDMs 110 and 120 are even closer to one another in the X-direction when the controller 1302 aligns each IDM to the virtual rail, thereby eliminating any relative misalignment between the robotic arms 110 and 120.

With reference to FIG. 13G, the controller 1302 moves the robotic arms 110 and 120 through a series of poses that simulate the insertion of a medical instrument, based on the further adjusted kinematic model, while the instruments 131 and 133 are coupled to the IDMs 115 and 125, respectively (such as described with reference to FIG. 13C). The controller 1302 also measures instrument driving torques τᵢ₁³ and τᵢ₂³ on the IDMs 115 and 125, respectively, or on motors that drive the IDMs during the instrument insertion. With reference to FIG. 13H, the controller 1302 moves the robotic arms 110 and 120 through a series of poses that simulate the retraction of a medical instrument, based on the further adjusted kinematic model, while the instruments 131 and 133 are coupled to the IDMs 115 and 125, respectively (such as described with reference to FIG. 13D). The controller 1302 also measures instrument driving torques τᵣ₁³ and τᵣ₂³ on the IDMs 115 and 125, respectively, or on motors that drive the IDMs during the instrument retraction.

In some implementations, the controller 1302 may compare the instrument driving torques with the baseline torques to determine whether the robotic arms 110 and 120 and/or the IDMs 115 and 125 are still misaligned (such as described with reference to FIG. 12). For example, the controller 1302 may determine that the IDMs 115 and 125 are relatively misaligned if |τᵢ₁⁰−τᵢ₁³|≥τₜₕ, if |τᵢ₂⁰−τᵢ₂³|≥τₜₕ, if |τᵣ₁⁰−τᵣ₁³|≥τₜₕ, and/or if |τᵣ₁⁰−τᵣ₂³|≥τₜₕ. In the example of FIGS. 13G and 13H, the controller 1302 may determine that the IDMs 115 and 125 are substantially aligned along the same axis in the Y-direction (and thus, the same virtual rail) based on comparing the instrument driving torques with the baseline torques for insertion and retraction of a medical instrument. Accordingly, the controller 1302 may store the current iteration (i=3) of the kinematic model to be used for subsequent medical procedures (and may refrain from performing any further adjustments to the kinematic model).

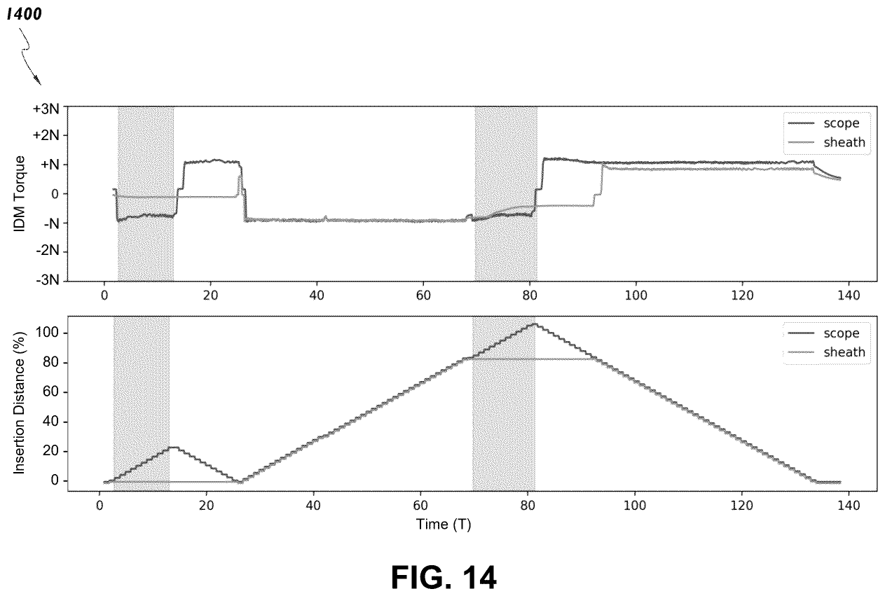

FIG. 14 shows another timing diagram 1400 depicting an example insertion and retraction of medical instruments coupled to robotic arms. In the example of FIG. 14, the medical instruments are shown to include a scope and a sheath. In some implementations, the sheath and the scope may be examples of the first instrument 131 and the second instrument 133, respectively, of FIGS. 13G and 13H. With reference for example to FIGS. 13G and 13H, the sheath may be coupled to the first robotic arm 110 and the scope may be coupled to the second robotic arm 120.

In the example of FIG. 14, IDM torque and insertion distance are described in relative terms, which may reflect any real-world values that can be recreated during insertion and retraction of a medical instrument. As described with reference to FIG. 11, IDM torque equal to “0” represents the resting torque of an IDM (without an instrument-driving forces being exerted or applied), which may correspond to a value greater or less than 0 Nm in actual procedures. Similarly, a 0% insertion distance reflects the position of the instruments at the start of the insertion/retraction operation depicted in FIG. 14, which may correspond to a value greater than 0 mm in actual procedures. Accordingly, the values depicted in FIG. 14 should not be construed as limiting the scope of this disclosure to any particular values and/or ranges of values.

In the example of FIG. 14, the scope is disposed within a lumen of the sheath while the robotic arms are relatively aligned according to an adjusted kinematic model (such as described with reference to FIGS. 13G and 13H). As a result, the scope is coaxial with the sheath. At time T=0, both the scope and the sheath are initially positioned at 0% insertion depth. Between times T=0 and T=25, the sheath remains stationary while the scope is inserted to an insertion depth≈20% (between times T=0 and T=12) and retracted back to 0% insertion depth (between times T=12 and T=25). Because the robotic arms relatively aligned, the insertion and retraction of the scope does not exert any misalignment torque on the IDMs coupled to the scope or the sheath.

As shown in FIG. 14, the IDM coupled to the sheath does not register any torque (≈0) while the scope is inserted and retracted between times T=0 and T=25. FIG. 14 also shows a relatively flat torque curve for the IDM coupled to the scope between times T=0 and T=12 (highlighted by gray bars). This is in contrast to FIG. 11, where the relative misalignment between the robotic arms results in a downward slope of the torque curve for the IDM driving insertion of the scope (such as between times T=70 and T=80). Further, as shown in FIG. 14, the absence of misalignment torque allows the scope to be driven to the insertion depth≈20% much faster than the in the example of FIG. 11.

Between times T=25 and T=70, the scope and the sheath are concurrently driven to an insertion depth≈90%. Then, between times T=70 and T=80, the scope is driven further to an insertion depth≈100% while the sheath remains stationary. FIG. 14 again shows a flat torque curve for the IDM coupled to the scope (highlighted by gray bars), between times T=70 and T=80, while driving to the maximum insertion depth. This is in contrast to FIG. 11, where the relative misalignment between the robotic arms results in a downward slope of the torque curve for the IDM driving insertion of the scope (such as between times T=175 and T=200). In the example of FIG. 14, the scope is retracted back to an insertion depth≈90% between times T=80 and T=90. Between times T=90 and T=135, the sheath and the scope are concurrently retracted back to 0% insertion depth.

As shown in FIG. 14, the adjusted kinematic model of FIGS. 13G and 13H corrects the relative misalignment between robotic arms so that the scope can be driven coaxially with the sheath during instrument insertion and retraction. Compared to a kinematic model that results in relative misalignment between the robotic arms (such as shown in FIG. 11), the adjusted kinematic model effectively reduces or eliminates any misalignment torque on the IDMs, thereby reducing the potential for damaging the instruments and/or overexerting the motors in the robotic arms when performing a medical procedure. The absence (or reduction) of misalignment torque also facilitates faster setup and completion of the medical procedure.

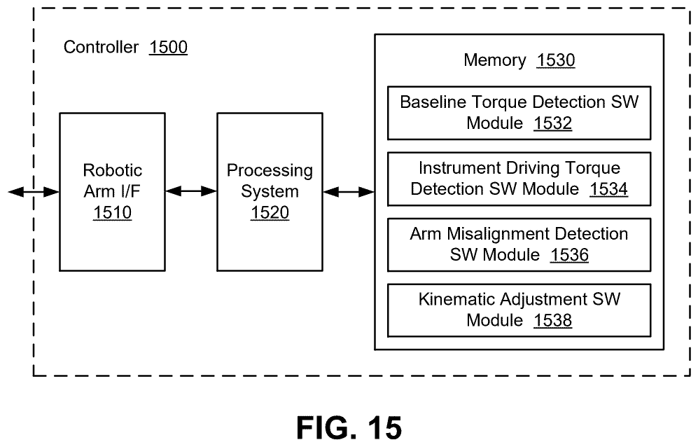

FIG. 15 shows a block diagram depicting an example controller 1500 for a robotic system, according to some implementations. In some implementations, the controller 1500 may be one example of the controller 1002 of FIGS. 10A and 10B or the controller 1302 of FIG. 13A-13H. More specifically, the controller 1500 is configured to control the poses and/or movements of two or more robotic arms of the robotic system and correct for relative misalignments between the robotic arms.

The controller 900 includes a robotic arm interface (I/F) 1510, a processing system 1520, and a memory 1530. The robotic arm interface 1510 is configured to communicate with the robotic arms, for example, by transmitting control signals to various motors associated with each robotic arm and/or receiving feedback from various sensors disposed in the robotic arms and/or the motors themselves. In some implementations, the control signals may control actuation of the motors to manipulate or maintain each robotic arm in a desired pose. In some implementations, the feedback may indicate torque and/or other forces exerted on or by the motors and/or joints in each robotic arm.

The memory 1530 may include a non-transitory computer-readable medium (including one or more nonvolatile memory elements, such as EPROM, EEPROM, Flash memory, or a hard drive, among other examples) that may store the following software (SW) modules: a baseline torque detection SW module 1532 to move a first robotic arm through a plurality of first poses based on a kinematic model while a first instrument is coupled to the first robotic arm, where the plurality of first poses cause the first instrument to be driven inside a lumen of a second instrument coupled to a second robotic arm, and to determine a torque on one or more motors of the first robotic arm in response to moving the first robotic arm through the plurality of first poses; an instrument driving torque detection SW module 1534 to move the first robotic arm through a plurality of second poses based on the kinematic model while the first instrument is decoupled from the first robotic arm and to determine a baseline torque on the one or more motors of the first robotic arm in response to moving the first robotic arm through the plurality of second poses; an arm misalignment detection SW module 1536 to determine whether the first robotic arm and the second robotic arm are misaligned based at least in part on the determined torque, and the baseline torque, on the one or more motors of the first robotic arm; and a kinematic adjustment SW module 1538 to selectively adjust the kinematic model based on whether the first robotic arm and the second robotic arm are misaligned. Each of the software modules 1532-1538 includes instructions that, when executed by the processing system 1520, causes the controller 1500 to perform the corresponding functions.

The processing system 1520 may include any suitable one or more processors capable of executing scripts or instructions of one or more software programs stored in the controller 1500 (such as in the memory 1530). For example, the processing system 1520 may execute the baseline torque detection SW module 1532 to move a first robotic arm through a plurality of first poses based on a kinematic model while a first instrument is coupled to the first robotic arm, where the plurality of first poses cause the first instrument to be driven inside a lumen of a second instrument coupled to a second robotic arm, and to determine a torque on one or more motors of the first robotic arm in response to moving the first robotic arm through the plurality of first poses. The processing system 1520 also may execute the instrument driving torque detection SW module 1534 to move the first robotic arm through a plurality of second poses based on the kinematic model while the first instrument is decoupled from the first robotic arm and to determine a baseline torque on the one or more motors of the first robotic arm in response to moving the first robotic arm through the plurality of second poses. The processing system 1520 may execute the arm misalignment detection SW module 1536 to determine whether the first robotic arm and the second robotic arm are misaligned based at least in part on the determined torque, and the baseline torque, on the one or more motors of the first robotic arm. The processing system 1520 may further execute the kinematic adjustment SW module 1538 to selectively adjust the kinematic model based on whether the first robotic arm and the second robotic arm are misaligned.

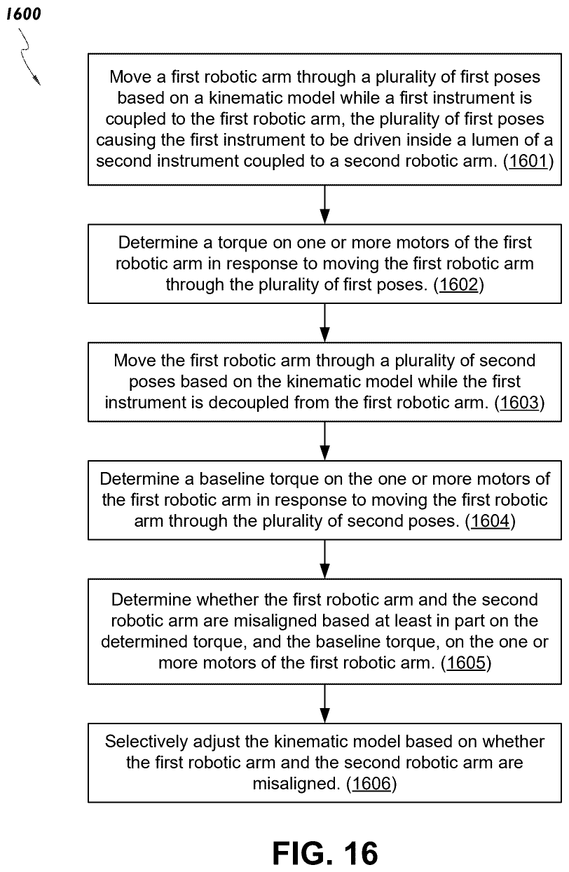

FIG. 16 shows an illustrative flowchart depicting an example operation 1600 for correcting relative misalignment between robotic arms, according to some implementations. In some implementations, the example operation 1600 may be performed by a controller for a robotic system such as the controller 1302 of FIG. 13A-13H or the controller 1500 of FIG. 15.

The controller may move a first robotic arm through a plurality of first poses based on a kinematic model while a first instrument is coupled to the first robotic arm, where the plurality of first poses causes the first instrument to be driven inside a lumen of a second instrument coupled to a second robotic arm (1601). The controller may determine a torque on one or more motors of the first robotic arm in response to moving the first robotic arm through the plurality of first poses (1602). The controller may move the first robotic arm through a plurality of second poses based on the kinematic model while the first instrument is decoupled from the first robotic arm (1603). The controller may determine a baseline torque on the one or more motors of the first robotic arm in response to moving the first robotic arm through the plurality of second poses (1604). The controller may determine whether the first robotic arm and the second robotic arm are misaligned based at least in part on the determined torque, and the baseline torque, on the one or more motors of the first robotic arm (1605). The controller may selectively adjust the kinematic model based on whether the first robotic arm and the second robotic arm are misaligned (1606).

In some implementations, the plurality of first poses may cause the first instrument to be inserted in the lumen of the second instrument. In some other implementations, the plurality of first poses may cause the first instrument to be retracted from the lumen of the second instrument. In some implementations, the plurality of second poses may mirror the plurality of first poses.

In some implementations, the determining of whether the first robotic arm and the second robotic arm are misaligned may include determining whether the determined torque on the one or more motors of the first robotic arm exceeds the baseline torque on the one or more motors of the first robotic arm by at least a threshold amount, and determining that the first robotic arm and the second robotic arm are misaligned responsive to determining that the torque on the one or more motors of the first robotic arm exceeds the baseline torque on the one or more motors of the first robotic arm by at least the threshold amount.

In some aspects, the controller may further move the second robotic arm through a plurality of third poses based on the kinematic model while the second instrument is coupled to the second robotic arm, where the plurality of third poses causes movement of the second instrument while the first instrument coupled to the first robotic arm is at least partially disposed within the lumen of the second instrument; determine a torque on one or more motors of the second robotic arm in response to moving the second robotic arm through the plurality of third poses; move the second robotic arm through a plurality of fourth poses based on the kinematic model while the second instrument is decoupled from the second robotic arm; and determine a baseline torque on the one or more motors of the second robotic arm in response to moving the second robotic arm through the plurality of fourth poses. In some implementations, the plurality of fourth poses may mirror the plurality of third poses.

In some implementations, the determining of whether the first robotic arm and the second robotic arm are misaligned may include determining whether the torque on the one or more motors of the second robotic arm exceeds the baseline torque on the one or more motors of the second robotic arm by at least a threshold amount, and determining that the first robotic arm and the second robotic arm are misaligned responsive to determining that the torque on the one or more motors of the second robotic arm exceeds the baseline torque on the one or more motors of the second robotic arm by at least the threshold amount.

In some aspects, the selective adjusting of the kinematic model may include adjusting the kinematic model responsive to determining that the first robotic arm and the second robotic arm are misaligned. In some implementations, the controller may further move the first robotic arm through a plurality of third poses based on the adjusted kinematic model while the first instrument is coupled to the first robotic arm, where the plurality of third poses causes the first instrument to be driven inside the lumen of the second instrument coupled to the second robotic arm; determine an updated torque on the one or more motors of the first robotic arm in response to moving the first robotic arm through the plurality of third poses; determine whether the first robotic arm and the second robotic arm are still misaligned based at least in part on the updated torque, and the baseline torque, on the one or more motors of the first robotic arm; and selectively adjust the adjusted kinematic model based on whether the first robotic arm and the second robotic arm are still misaligned.

In some other implementations, the controller may further move the second robotic arm through a plurality of third poses based on the adjusted kinematic model while the second instrument is coupled to the second robotic arm, where the plurality of third poses causes movement of the second instrument while the first instrument coupled to the first robotic arm is at least partially disposed within the lumen of the second instrument; determine an updated torque on the one or more motors of the second robotic arm in response to moving the second robotic arm through the plurality of third poses; determine whether the first robotic arm and the second robotic arm are still misaligned based at least in part on the updated torque, and the baseline torque, on the one or more motors of the second robotic arm; and selectively adjust the adjusted kinematic model based on whether the first robotic arm and the second robotic arm are still misaligned.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure.

Thus, the claims are not intended to be limited to the implementations shown herein, but are to be accorded the widest scope consistent with this disclosure, the principles and the novel features disclosed herein.

What is claimed is:

1. A method performed by a robotic system, comprising:
moving a first robotic arm through a plurality of first poses based on a kinematic model while a first instrument is coupled to the first robotic arm, the plurality of first poses causing the first instrument to be driven inside a lumen of a second instrument coupled to a second robotic arm;
determining a torque on one or more motors of the first robotic arm in response to moving the first robotic arm through the plurality of first poses;
moving the first robotic arm through a plurality of second poses based on the kinematic model while the first instrument is decoupled from the first robotic arm;
determining a baseline torque on the one or more motors of the first robotic arm in response to moving the first robotic arm through the plurality of second poses;
determining whether the first robotic arm and the second robotic arm are misaligned based at least in part on the determined torque, and the baseline torque, on the one or more motors of the first robotic arm; and
selectively adjusting the kinematic model based on whether the first robotic arm and the second robotic arm are misaligned.

2. The method of claim 1, wherein the plurality of first poses causes the first instrument to be inserted in the lumen of the second instrument.

3. The method of claim 1, wherein the plurality of first poses causes the first instrument to be retracted from the lumen of the second instrument.

4. The method of claim 1, wherein the plurality of second poses mirrors the plurality of first poses.

5. The method of claim 1, wherein the determining of whether the first robotic arm and the second robotic arm are misaligned comprises:
determining whether the torque on the one or more motors of the first robotic arm exceeds the baseline torque on the one or more motors of the first robotic arm by at least a threshold amount; and
determining that the first robotic arm and the second robotic arm are misaligned responsive to determining that the torque on the one or more motors of the first robotic arm exceeds the baseline torque on the one or more motors of the first robotic arm by at least the threshold amount.

6. The method of claim 1, further comprising:
moving the second robotic arm through a plurality of third poses based on the kinematic model while the second instrument is coupled to the second robotic arm, the plurality of third poses causing movement of the second instrument while the first instrument coupled to the first robotic arm is at least partially disposed within the lumen of the second instrument;
determining a torque on one or more motors of the second robotic arm in response to moving the second robotic arm through the plurality of third poses;
moving the second robotic arm through a plurality of fourth poses based on the kinematic model while the second instrument is decoupled from the second robotic arm; and
determining a baseline torque on the one or more motors of the second robotic arm in response to moving the second robotic arm through the plurality of fourth poses.

7. The method of claim 6, wherein the plurality of fourth poses mirrors the plurality of third poses.

8. The method of claim 6, wherein the determining of whether the first robotic arm and the second robotic arm are misaligned comprises:
determining whether the torque on the one or more motors of the second robotic arm exceeds the baseline torque on the one or more motors of the second robotic arm by at least a threshold amount; and
determining that the first robotic arm and the second robotic arm are misaligned responsive to determining that the torque on the one or more motors of the second robotic arm exceeds the baseline torque on the one or more motors of the second robotic arm by at least the threshold amount.

9. The method of claim 1, wherein the selective adjusting of the kinematic model comprises:
adjusting the kinematic model responsive to determining that the first robotic arm and the second robotic arm are misaligned.

10. The method of claim 9, further comprising:
moving the first robotic arm through a plurality of third poses based on the adjusted kinematic model while the first instrument is coupled to the first robotic arm, the plurality of third poses causing the first instrument to be driven inside the lumen of the second instrument coupled to the second robotic arm;
determining an updated torque on the one or more motors of the first robotic arm in response to moving the first robotic arm through the plurality of third poses;
determining whether the first robotic arm and the second robotic arm are still misaligned based at least in part on the updated torque, and the baseline torque, on the one or more motors of the first robotic arm; and
selectively adjusting the adjusted kinematic model based on whether the first robotic arm and the second robotic arm are still misaligned.

11. The method of claim 9, further comprising:
moving the second robotic arm through a plurality of third poses based on the adjusted kinematic model while the second instrument is coupled to the second robotic arm, the plurality of third poses causing movement of the second instrument while the first instrument coupled to the first robotic arm is at least partially disposed within the lumen of the second instrument;
determining an updated torque on the one or more motors of the second robotic arm in response to moving the second robotic arm through the plurality of third poses;
determining whether the first robotic arm and the second robotic arm are still misaligned based at least in part on the updated torque, and the baseline torque, on the one or more motors of the second robotic arm; and
selectively adjusting the adjusted kinematic model based on whether the first robotic arm and the second robotic arm are still misaligned.

12. A controller for a robotic system comprising:
a processing system; and
a memory storing instructions that, when executed by the processing system, cause the controller to:
move a first robotic arm through a plurality of first poses based on a first kinematic model while a first instrument is coupled to the first robotic arm, the plurality of first poses causing the first instrument to be driven inside a lumen of a second instrument coupled to a second robotic arm;
determine a torque on one or more motors of the first robotic arm in response to moving the first robotic arm through the plurality of first poses;
move the first robotic arm through a plurality of second poses based on the first kinematic model while the first instrument is decoupled from the first robotic arm;
determine a baseline torque on the one or more motors of the first robotic arm in response to moving the first robotic arm through the plurality of second poses;
determine whether the first robotic arm and the second robotic arm are misaligned based at least in part on the determined torque, and the baseline torque, on the one or more motors of the first robotic arm; and
selectively adjust the first kinematic model or a second kinematic model associated with the second robotic arm based on whether the first robotic arm and the second robotic arm are misaligned.

13. The controller of claim 12, wherein the plurality of second poses mirrors the plurality of first poses.

14. The controller of claim 12, wherein the determining of whether the first robotic arm and the second robotic arm are misaligned comprises:
determining whether the torque on the one or more motors of the first robotic arm exceeds the baseline torque on the one or more motors of the first robotic arm by at least a threshold amount; and
determining that the first robotic arm and the second robotic arm are misaligned responsive to determining that the torque on the one or more motors of the first robotic arm exceeds the baseline torque on the one or more motors of the first robotic arm by at least the threshold amount.

15. The controller of claim 12, wherein execution of the instructions further causes the controller to:
move the second robotic arm through a plurality of third poses based on the kinematic model while the second instrument is coupled to the second robotic arm, the plurality of third poses causing movement of the second instrument while the first instrument coupled to the first robotic arm is at least partially disposed within the lumen of the second instrument;
determine a torque on one or more motors of the second robotic arm in response to moving the second robotic arm through the plurality of third poses;
move the second robotic arm through a plurality of fourth poses based on the kinematic model while the second instrument is decoupled from the second robotic arm; and
determine a baseline torque on the one or more motors of the second robotic arm in response to moving the second robotic arm through the plurality of fourth poses.

16. The controller of claim 15, wherein the determining of whether the first robotic arm and the second robotic arm are misaligned comprises:
determining whether the torque on the one or more motors of the second robotic arm exceeds the baseline torque on the one or more motors of the second robotic arm by at least a threshold amount; and
determining that the first robotic arm and the second robotic arm are misaligned responsive to determining that the torque on the one or more motors of the second robotic arm exceeds the baseline torque on the one or more motors of the second robotic arm by at least the threshold amount.

17. The controller of claim 12, wherein the selective adjusting of the kinematic model comprises:
adjusting the kinematic model responsive to determining that the first robotic arm and the second robotic arm are misaligned.

18. The controller of claim 17, wherein execution of the instructions further causes the controller to:
move the first robotic arm through a plurality of third poses based on the adjusted kinematic model while the first instrument is coupled to the first robotic arm, the plurality of third poses causing the first instrument to be driven inside the lumen of the second instrument coupled to the second robotic arm;
determine an updated torque on the one or more motors of the first robotic arm in response to moving the first robotic arm through the plurality of third poses;
determine whether the first robotic arm and the second robotic arm are still misaligned based at least in part on the updated torque, and the baseline torque, on the one or more motors of the first robotic arm; and
selectively adjust the adjusted kinematic model based on whether the first robotic arm and the second robotic arm are still misaligned.

19. The controller of claim 17, wherein execution of the instructions further causes the controller to:
move the second robotic arm through a plurality of third poses based on the adjusted kinematic model while the second instrument is coupled to the second robotic arm, the plurality of third poses causing movement of the second instrument while the first instrument coupled to the first robotic arm is at least partially disposed within the lumen of the second instrument;
determine an updated torque on the one or more motors of the second robotic arm in response to moving the second robotic arm through the plurality of third poses;
determine whether the first robotic arm and the second robotic arm are still misaligned based at least in part on the updated torque, and the baseline torque, on the one or more motors of the second robotic arm; and
selectively adjust the adjusted kinematic model based on whether the first robotic arm and the second robotic arm are still misaligned.

20. A system comprising:
a first robotic arm;
a second robotic arm; and
a controller configured to:
move the first robotic arm through a plurality of first poses based on a first kinematic model while a first instrument is coupled to the first robotic arm, the plurality of first poses causing the first instrument to be driven inside a lumen of a second instrument coupled to the second robotic arm;
determine a torque on one or more motors of the first robotic arm in response to moving the first robotic arm through the plurality of first poses;
move the first robotic arm through a plurality of second poses based on the first kinematic model while the first instrument is decoupled from the first robotic arm;
determine a baseline torque on the one or more motors of the first robotic arm in response to moving the first robotic arm through the plurality of second poses;
determine whether the first robotic arm and the second robotic arm are misaligned based at least in part on the determined torque, and the baseline torque, on the one or more motors of the first robotic arm; and
selectively adjust the first kinematic model or a second kinematic model associated with the second robotic arm based on whether the first robotic arm and the second robotic arm are misaligned.
